(19) United States
(12) Patent Application Publication
(10) Pub. No.: US 2011/0152632 A1
(43) Pub. Date: Jun. 23, 2011

(54) Universal Body Sensor Network

(75) Inventors: Olivier Le Neel, Singapore (SG); Mehdi Khaled, Singapore (SG)

(73) Assignee: E-VITAE PTE. LTD., Singapore (SG)

(21) Appl. No.: 13/057,985

(22) PCT Filed: Aug. 6, 2009
(86) PCT No.: PCT/IB2009/053428

§ 371 Date: Mar. 13, 2011

(30) Foreign Application Priority Data
Aug. 6, 2008 (SG) 200805849-7
Apr. 27, 2009 (SG) 200902874-7

(51) Int. Cl. A61B 5/00

(52) U.S. Cl. 600/300

(57) ABSTRACT

A sensor module (2A00, 2B00, 2C00) for an on-body sensor (1A00, 1B00) is provided. The sensor (1A00, 1B00) is intended for connecting to the sensor module (2A00, 2B00, 2C00) and for transmitting an analog value of a patient (18). The sensor module (2A00, 2B00, 2C00) comprises a converter means, a storage means, a determining means, and a transmitting means. The converter means (37) converts the analog value of the patient (18) to its digital format whilst the storage means (40, 41) stores the digital value. The determining means (35) determines a parameter of the patient (18) using a value of the digital value to and the transmitting means (39) sends data regarding the parameter.

This application relates to a personal area network.

In medical applications, the personal area network includes wireless means for communicating between medical devices and computing devices.

The medical device includes implants that may be located within or at a human body of a living individual whilst the computing device comprises computers that may be located outside or on a surface of the human body. The medical device can also include data sensors, such as heart or brain monitors. The data sensors can be provided inside the human body or at its surface for communicating with data receivers and with computers that are located outside the human body so that medical or equally qualified personnel may review a particular condition of the human body within its immediate environment through an analysis of collected data.

Pairings of the sensor and the computing device may seemingly require only one-way broadcast of information. However, a Body Area Network (BAN), which includes the data sensors, enjoys certain benefits in using a two-way communication to operate with the computing device.

For example, the BAN may send information to a receiver or to a computer system. The two-way communication can be used for correcting data communication errors, for managing timings of data transfers, and for verifying that the medical device is properly operating. The two-way communication can also be used for management of energy resources through controlling a certain device mode—but not limited to—between a stand-by mode or in an active mode using device commands.

The application provides a sensor module for an on-body sensor of a Body Area Network.

It is believed that the sensor module can provides a means for adapting different type of sensors to a sensor hub. Further, the sensor module can be configured by software for easier adaption. New sensor may also be to work with the sensor module with only a change of the sensor module software.

The sensor is intended for connecting to the sensor module and for transmitting an analog signal of a body of a patient to the sensor module. The analog signal can be related to voltage, electrical current, or capacitance.

The sensor module comprises a converter means for converting the analog signal to a digital signal. This prevents the digital signal from losses due to resistance. Further, the digital signal enables application of digital signal techniques.

In addition, the sensor module includes a storage means for storing the digital signal for later processing.

The sensor module also includes a determining means for determining or identifying a parameter of the patient using a value of the digital signal. The parameter can be related to a biological, electrophysiological, or physical characteristic of the patient. For example, the patient parameter can be related a patient temperature or a patient glucose concentration level. The determination can use a simple offset or a relationship graph of the patient parameter with respect to the digital signal value. A manufacturer of the sensor can provide the graph.

Further, the sensor module includes a transmitting means for sending data regarding the patient parameter to another device. The data can be sent to a sensor hub for later trans-mission to medical personnel.

For reducing signal distortion, an amplifier can be included in the sensor module for amplifying the analog signal before the analog signal is converted to the digital signal. The conversion may introduce signal noise. However, the signal amplification would reduce a signal to noise ratio.

The determining means can comprise a processor for determining the parameter of the patient whilst a software program directs or guides the processor. The software program may include firmware and data, which are sent by the sensor hub. The sensor hub selects the appropriate firmware and data that is appropriate for the sensor. A different software program can be loaded or installed into the sensor module when a different sensor is connected to the sensor module. Put differently, the sensor module can be adapted or configured for different sensor or different sensor type.

The storage means can also comprise a computer memory. The computer memory can also include different types of computer memory, such as the ROM (Read Only Memory), the DRAM (Dynamic Random Access Memory, or the EPROM (Electrically Programmable Read Only Memory).

The computer memory can comprise or store data regarding sensor identifier for later identifying or tagging of the data regarding the patient parameter. This allows the data to be differentiated from data of other patient.

The sensor module can also include a clock for recording a timing of the analog signal. The timing can be related to time of receipt of the analog signal by the sensor module. Information about the timing can be later incorporated into the data regarding patient parameter.

Further, the sensor module may include a device for providing electrical power to the sensor. The sensor usually needs electrical power to function properly. The device may receive the electrical power from a power source, such a button battery, or from an external source.

The transmitting means can comprise a means for wired trans-mission or wireless transmission of the data regarding the patient parameter. The wired means can also serve as a channel for receiving external electrical power to the sensor module.

The application also provides a monitoring device of a Body Area Network. The monitoring device comprises a sensor module that is described above and a sensor that is also connected to the sensor module.

The application also provides a sensor hub for receiving a pre-determined signal from one or more monitoring devices that are described above. The pre-determined signal may include data regarding a parameter of a patient. The sensor hub processes the pre-determined signal. The process may detect signal abnormality. The sensor hub also sends the processed signal to a wrist monitor for display or alert.

The application also provides a universal Body Area Network that comprises one or more monitoring devices and a sensor hub that is connected to the one or more monitoring devices. The one or more monitoring devices and the sensor hub are described above.

The sensor hub may include a means for providing a periodic summary of the patient parameter for the patient so that the patient or the medical personnel can track easily a condition of the patient.

The application provides a method of operating a sensor module for an on-body sensor of a Body Area Network.

The method comprises the step of the sensor transmitting an analog signal of a body of a patient to the sensor module. Then, the sensor mode converts the analog signal of the patient is a digital signal and it afterward stores the digital signal. A parameter of the patient is later determined from the digital signal. The parameter can be related to a biological, electrophysiological, or physical parameter of the patient. Data regarding the determined parameter is afterward sent to an external device, such as a sensor hub.

The analog signal can be amplified before the analog signal is converted to the digital signal. Data regarding sensor identifier can be incorporated into the data regarding the patient parameter for identifying or tagging the sensor to the patient. A timing of the digital signal can also be recorded and be incorporated into the patient parameter data. An electrical power can also be provided to the sensor.

The application provides a method to collect, compile, exchange, and analyze clinical and non-clinical information. The method supports and enhances an accuracy of clinical decision. The method comprises the method of operating a sensor module that is described above.

The application provides a Body Area Network, which may comprise multiple sensors with corresponding sensor modules.

The sensor modules may fulfill different data collection purposes and features. Furthermore, the sensor modules treat data measured by the sensors at points of measurement, instead of a simple data collection, storage, and posting of raw data, as generally performed in such application. The data treatment includes conversion of the measured data from an analog format to a digital format. The sensor modules may also compute averages of measurements of interest and then prepare a data or relevant mathematical treatment that is appropriate to the sensor of interest and sensor module goal. The converted data may then be sent to a portable sensor hub for storage, analysis, and other data handling features.

The sensor module function can be adapted to support different types of data collected through the sensors. This can be accomplished by an installation of a sensor support algorithm in the sensor module during an initialization phase of the sensor module. This algorithm includes a specific computation rules and mathematical treatment adapted to a purpose of the sensor of interest.

In addition, the sensor modules have the advantage of scalability. The number of required sensor modules can be changed to fulfill various medical requirements.

The device of the application may be indicated or used by qualified medical personnel to gather, compile, analyze, and transfer combined physiological and non-physiological data to support clinical decision-making of particular medical conditions.

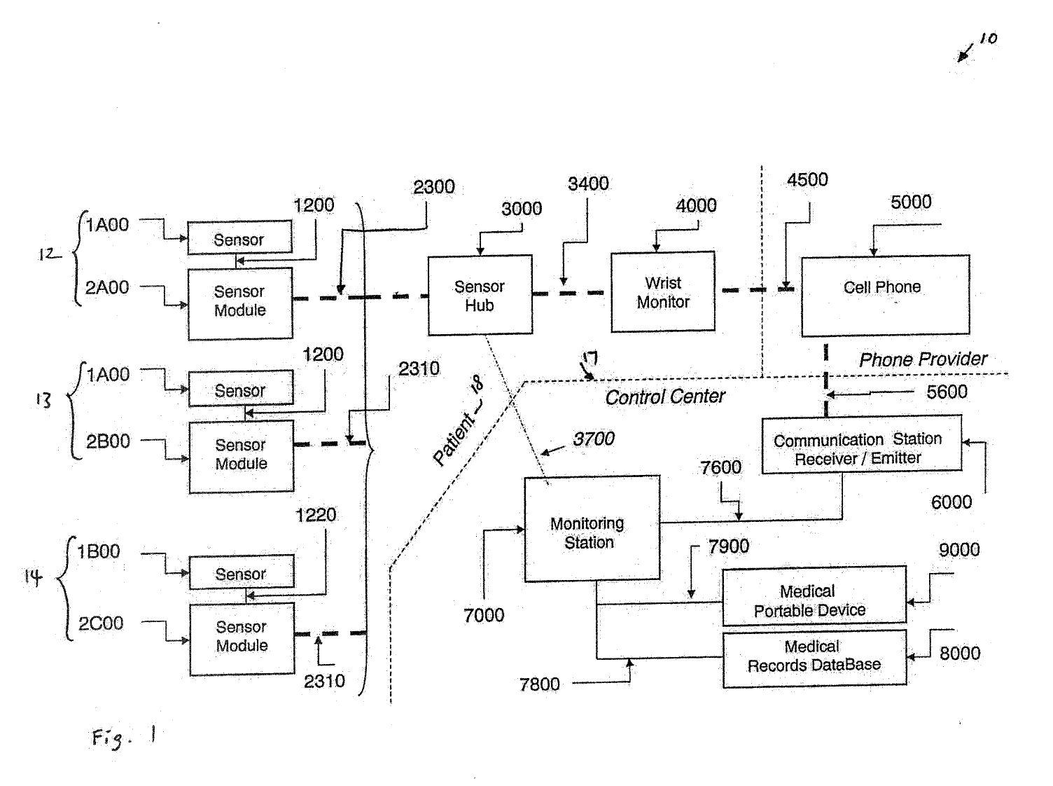

FIG. 1 illustrates an embodiment of a Universal Body Area Network that includes Universal Body Unit Sensors (u-BUS),

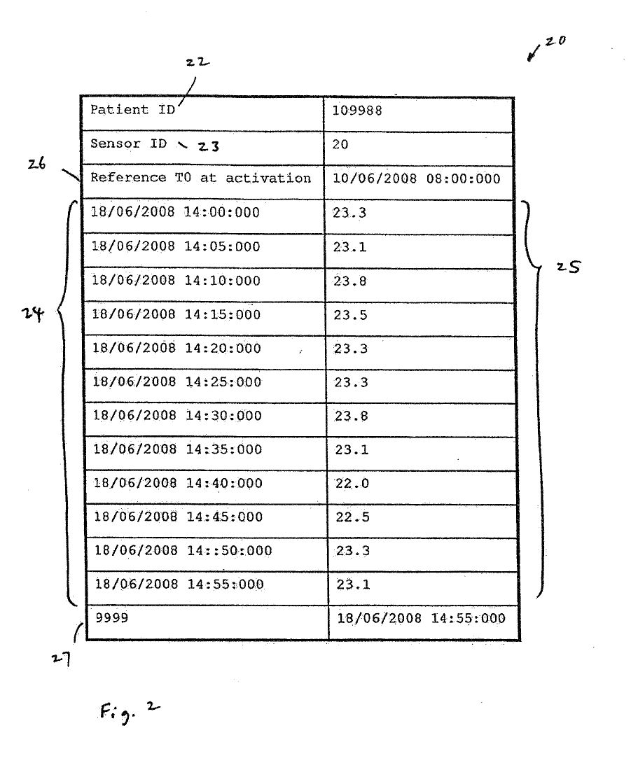

FIG. 2 illustrates an embodiment of a format of information of the u-BUS of FIG. 1,

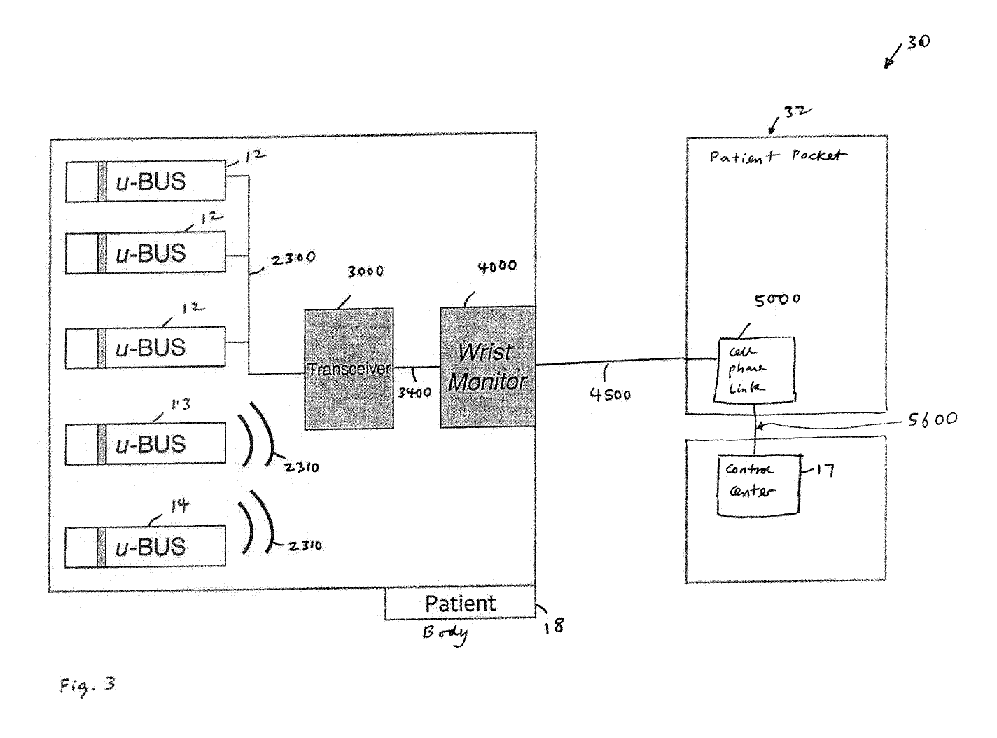

FIG. 3 illustrates a further embodiment of the Universal Body Area Network of FIG. 1,

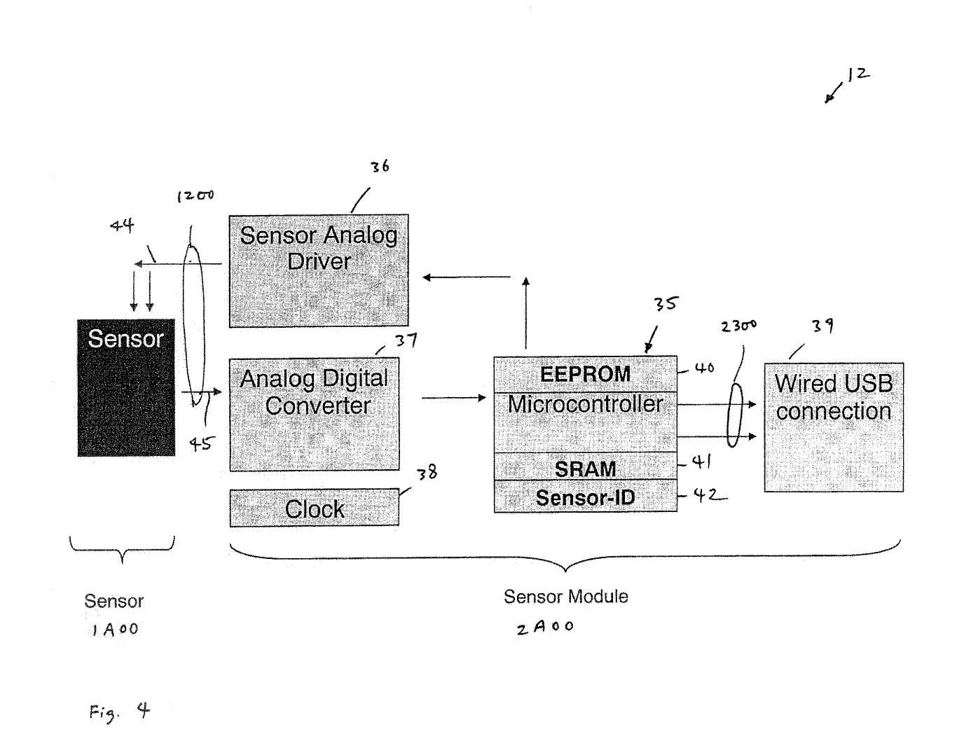

FIG. 4 illustrates an embodiment of the u-BUS of FIG. 1 or 3 that has a wired interface,

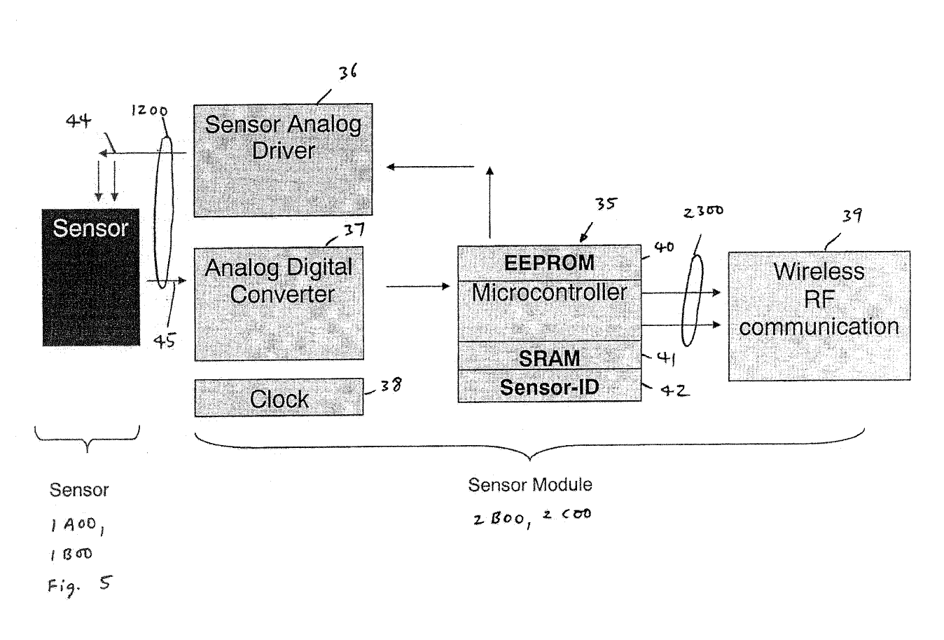

FIG. 5 illustrates a further embodiment of the u-BUS of FIG. 1 that has a wireless interface,

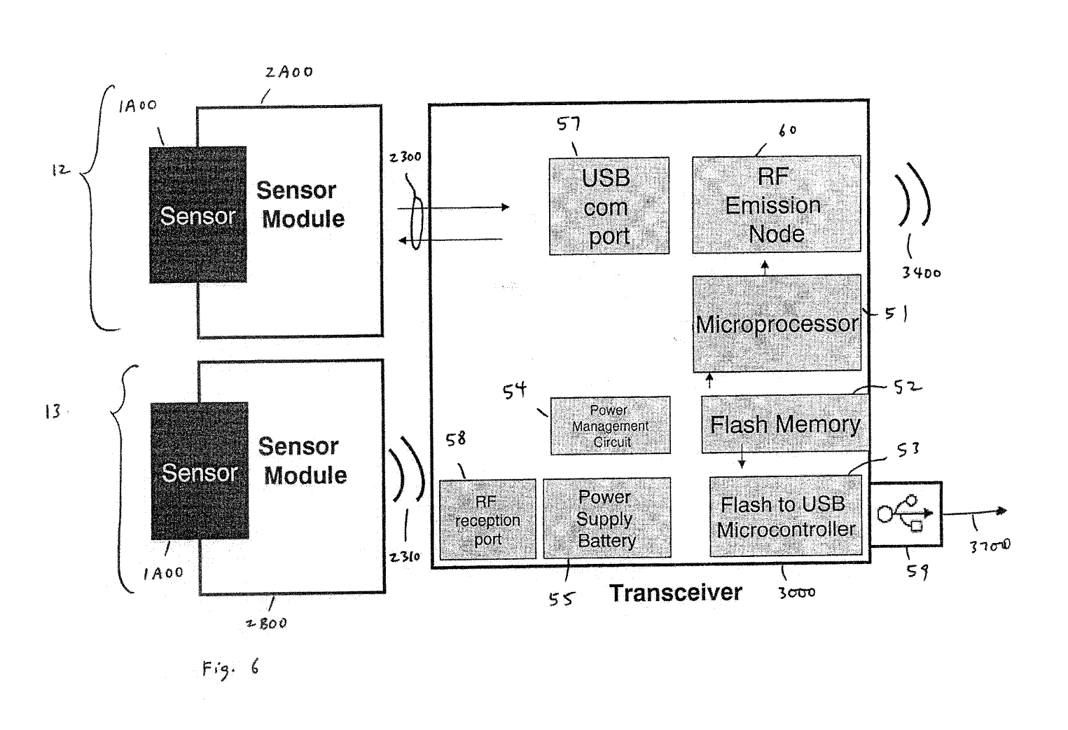

FIG. 6 illustrates an embodiment of a transceiver of the Universal Body Area Network of FIG. 1,

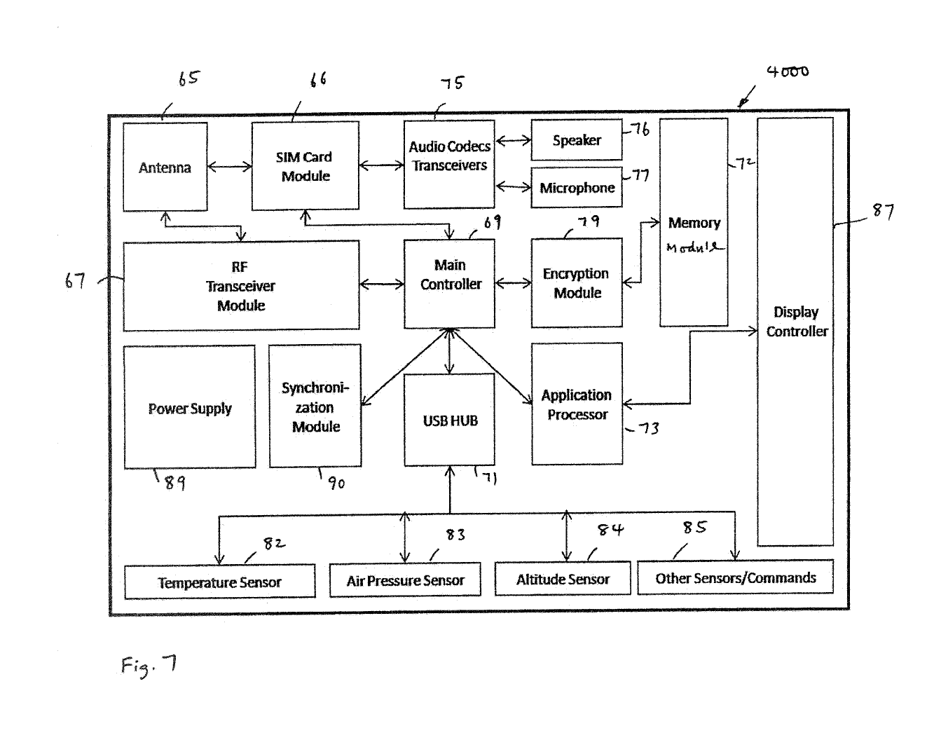

FIG. 7 illustrates an embodiment of a wrist monitor of the Universal Body Area Network of FIG. 1,

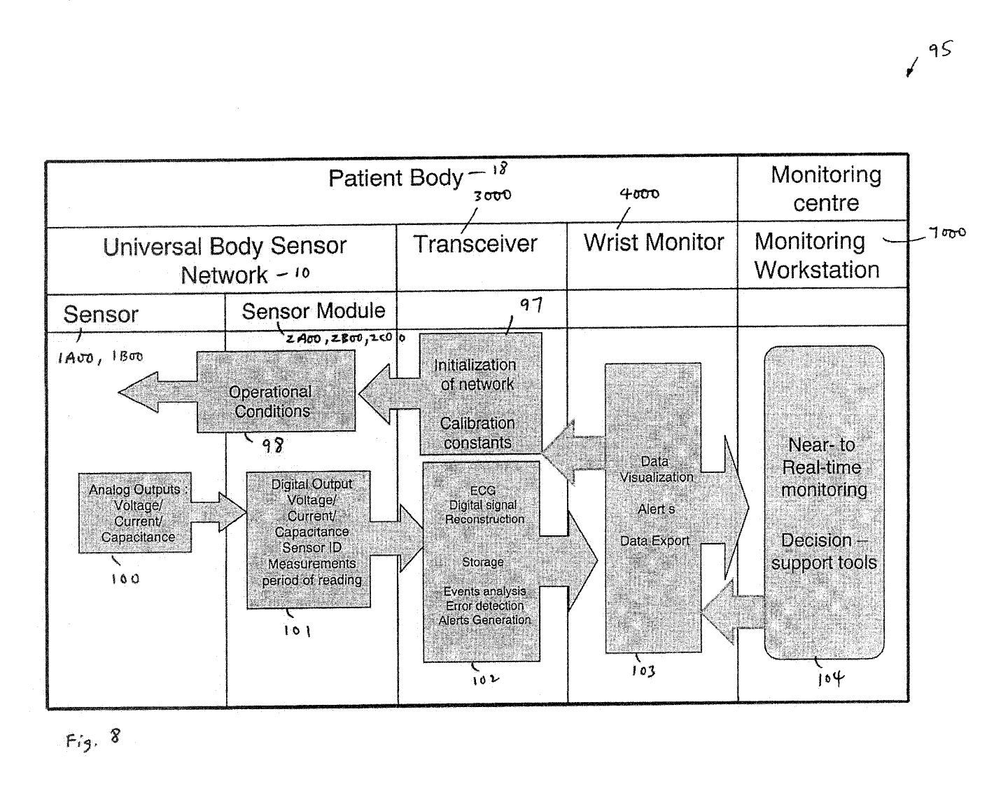

FIG. 8 illustrates an embodiment of a communication flow chart of the Universal Body Area Network of FIG. 1,

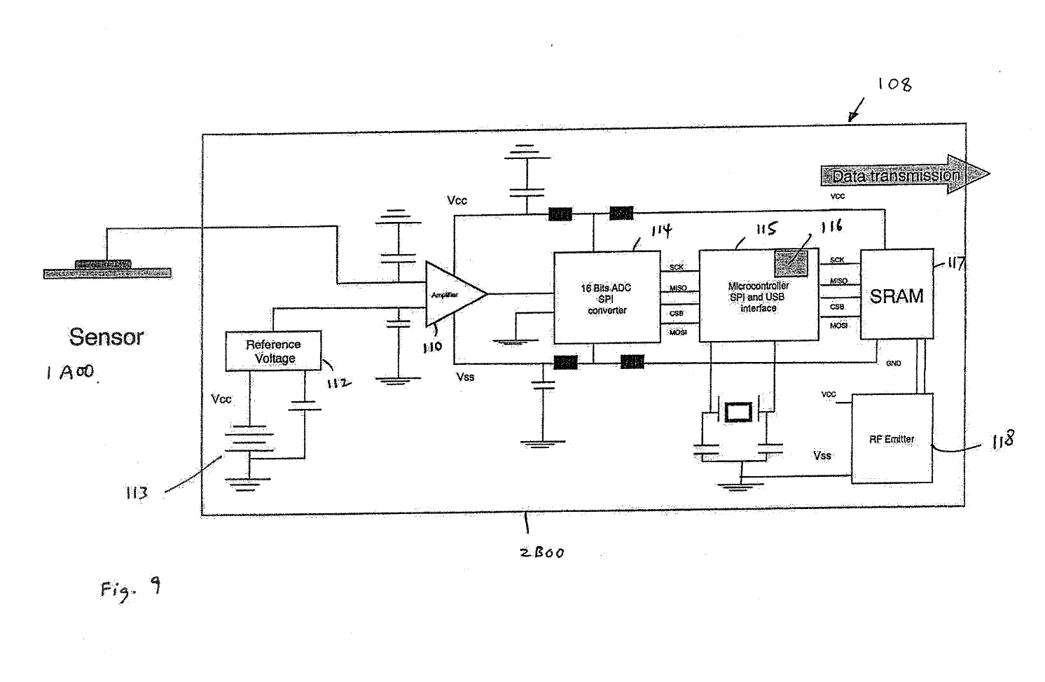

FIG. 9 illustrates an embodiment a schematic diagram of the U-BUS of FIG. 1,

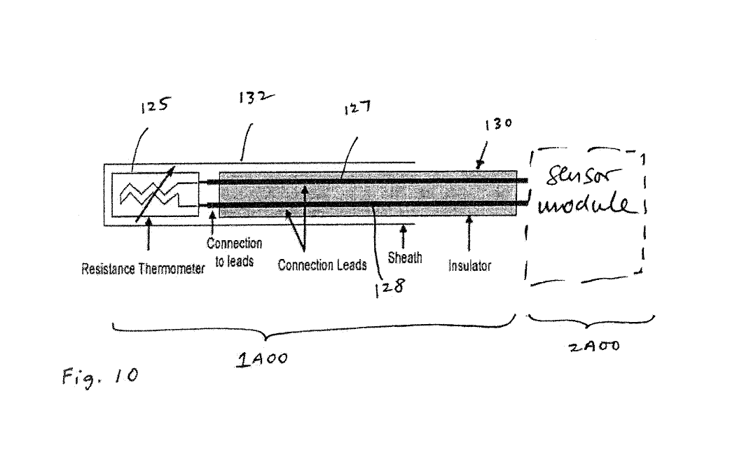

FIG. 10 illustrates an embodiment of the sensor of the U-BUS of FIG. 1,

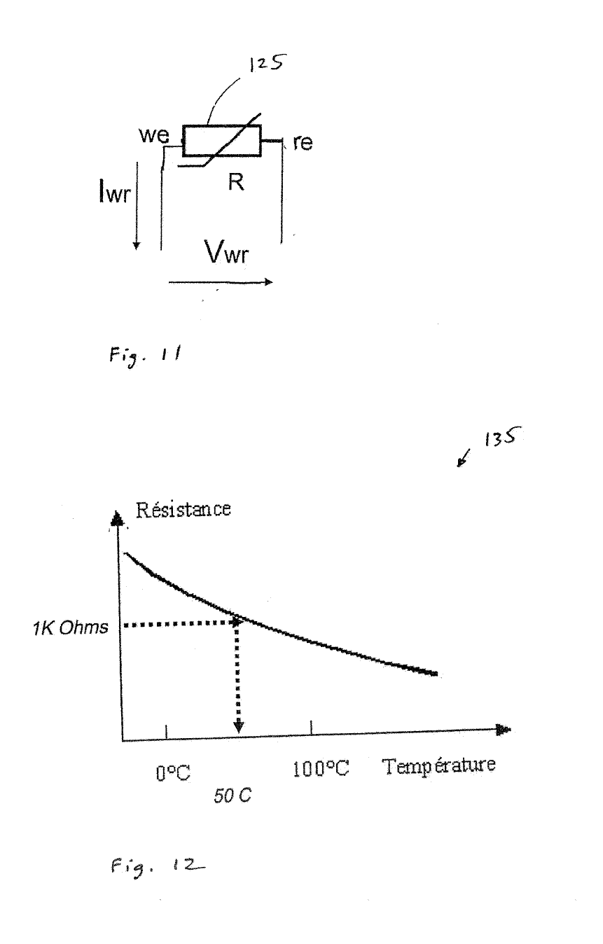

FIG. 11 illustrates a schematic diagram the sensor of FIG. 10,

FIG. 12 illustrates a response graph of the sensor of FIG. 10,

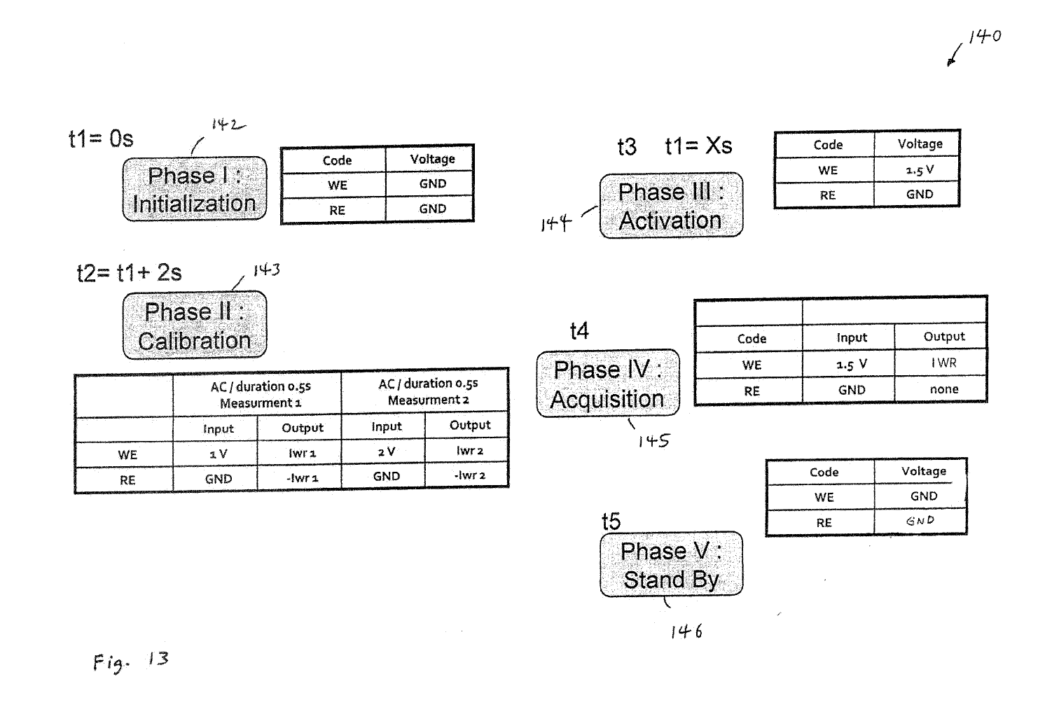

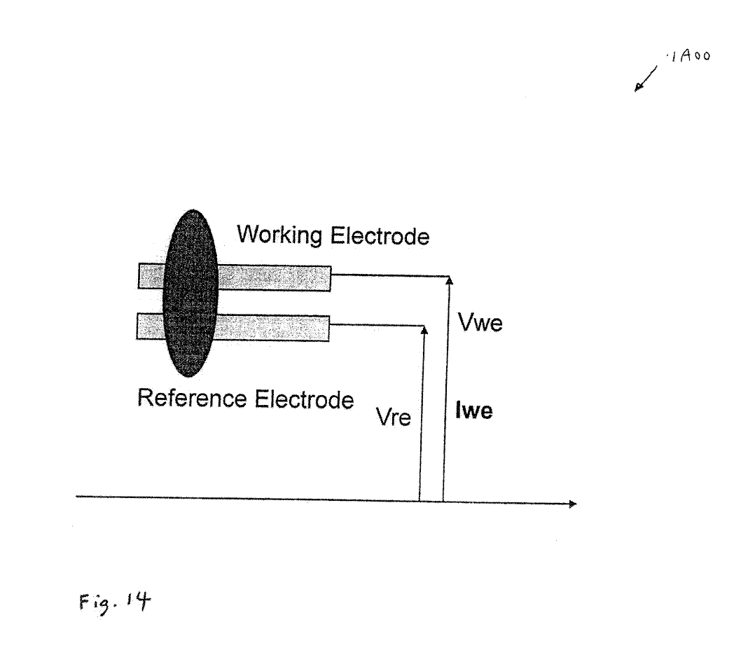

FIG. 13 illustrates a flow chart of the sensor of FIG. 10,

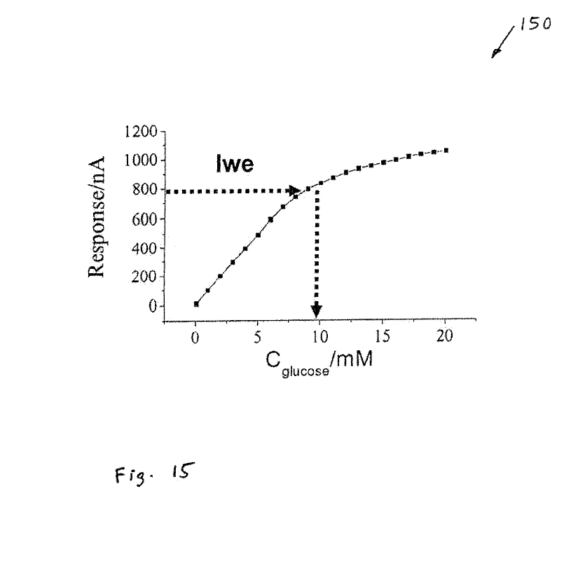

FIG. 14 illustrates a further embodiment of the sensor of the U-BUS of FIG. 1,

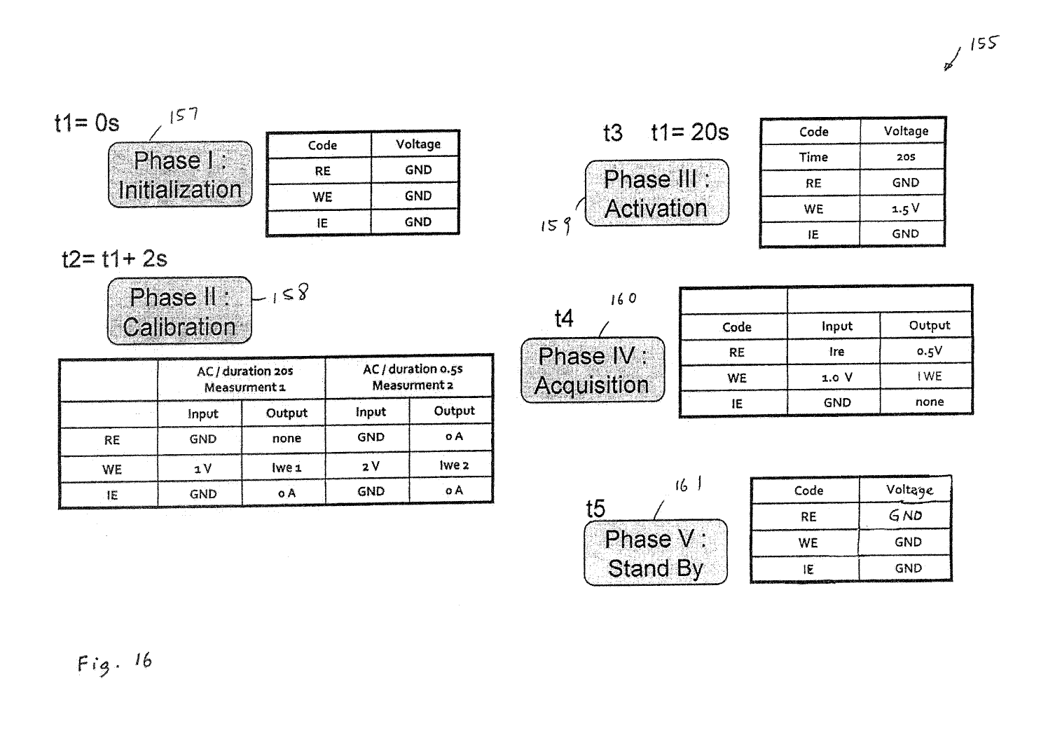

FIG. 15 illustrates a response graph of the sensor of FIG. 14,

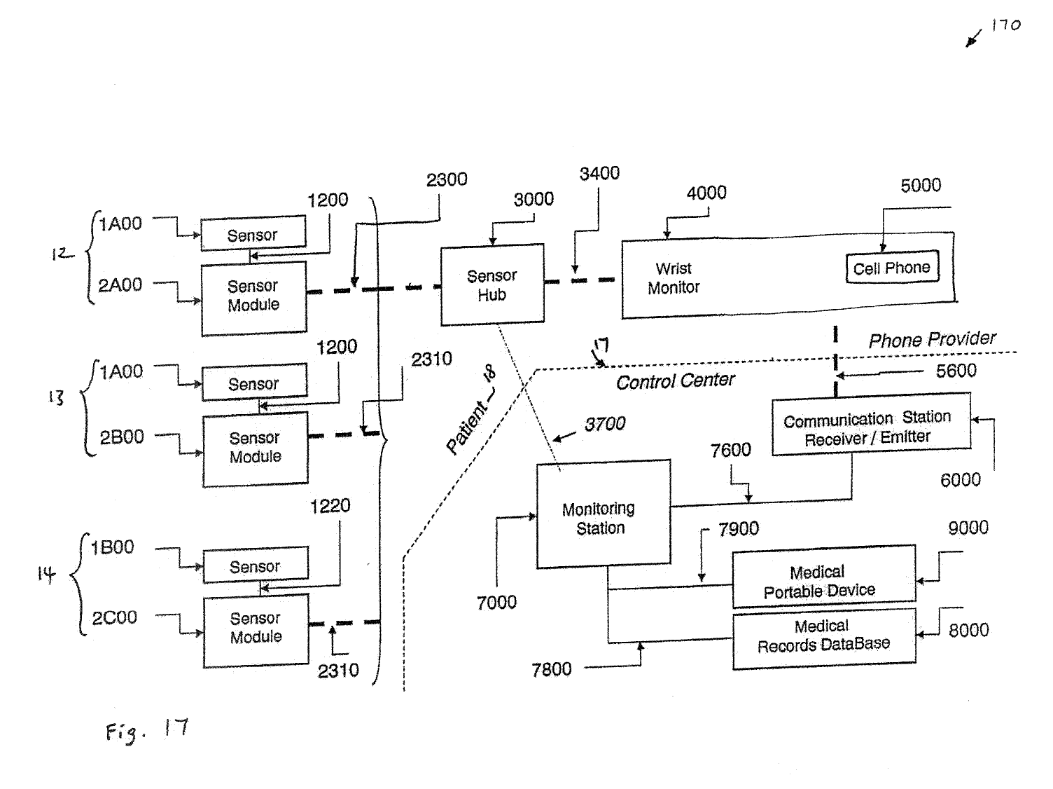

FIG. 16 illustrates a flow chart of the sensor of FIG. 14,

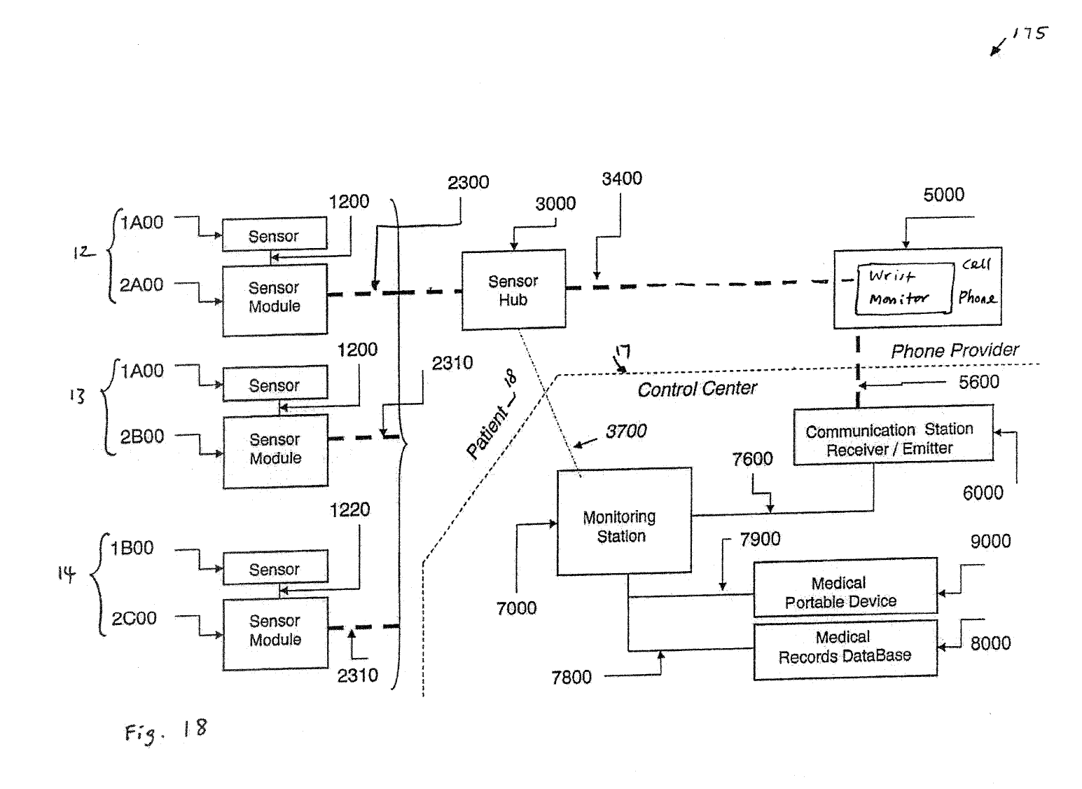

FIG. 17 illustrates a further embodiment of the Universal Body Area Network of FIG. 1,

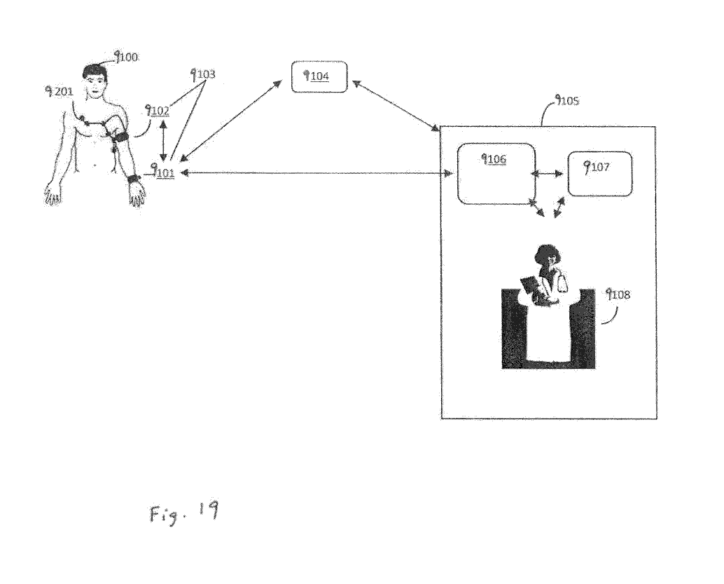

FIG. 18 illustrates another embodiment of the Universal Body Area Network of FIG. 1,

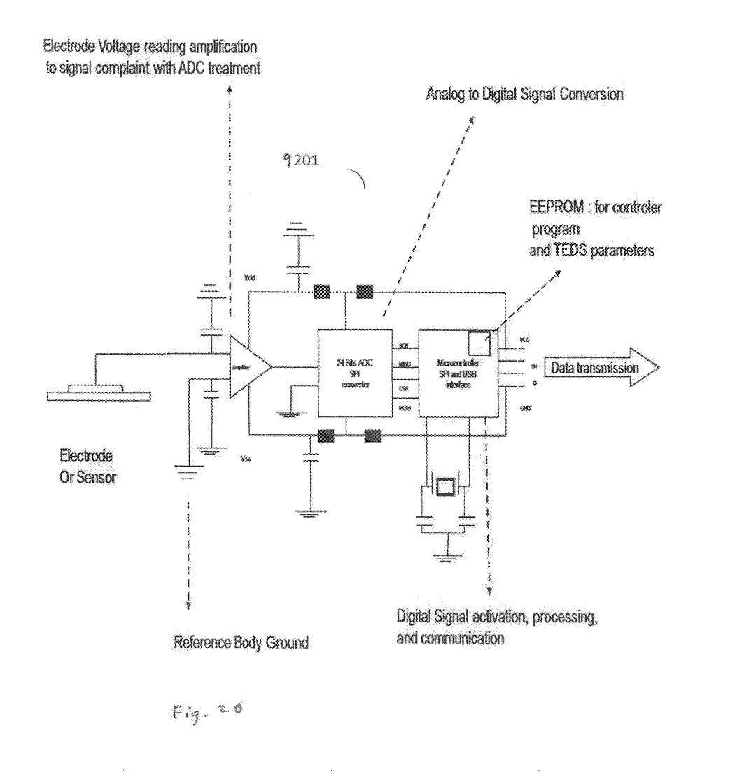

FIG. 19 illustrates, according to an exemplary embodiment, a method for bidirectional communication, monitoring and reporting physiological and non-physiological events, as well as human interaction,

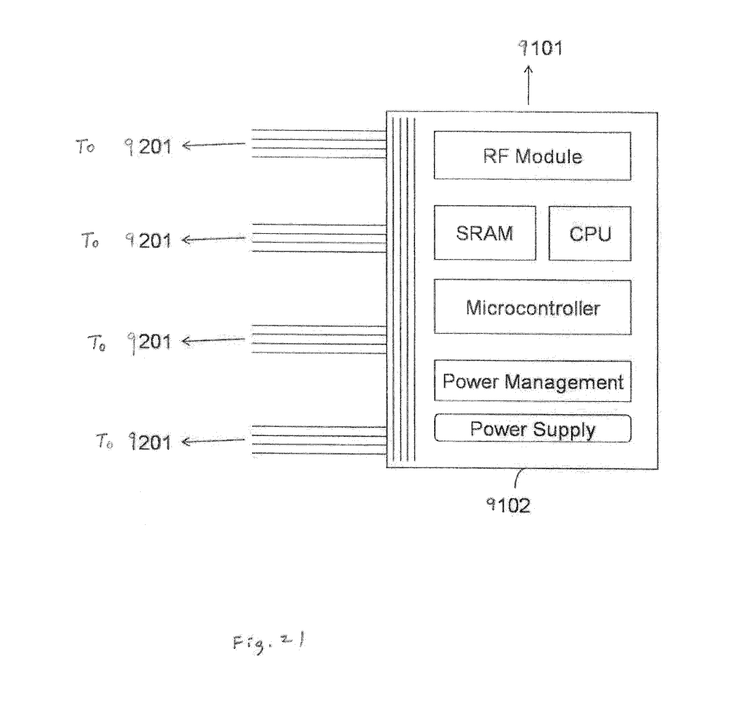

FIG. 20 illustrates a method for signal pick-up amplification and processing,

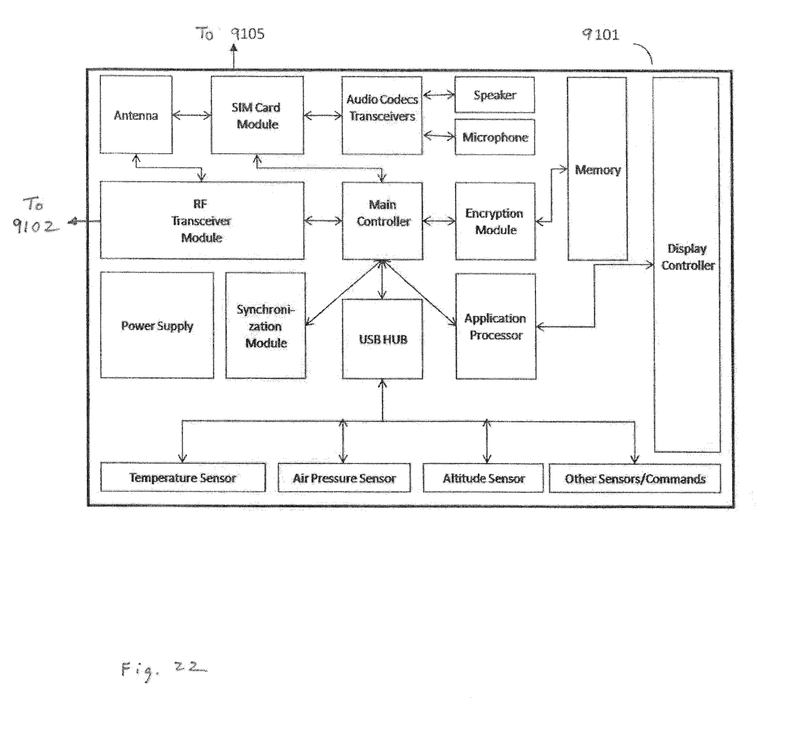

FIG. 21 illustrates a diagram, according to another embodiment, a procedure for monitoring, assessing, and processing the physiological and non-physiological information,

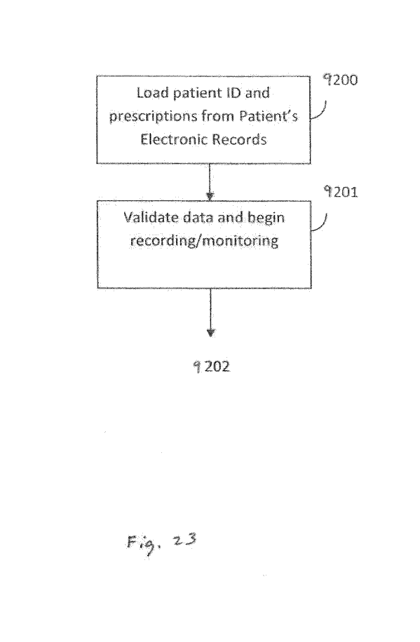

FIG. 22 illustrates a diagram, according to another embodiment, showing a procedure for monitoring, assessing, and processing the physiological and non-physiological information,

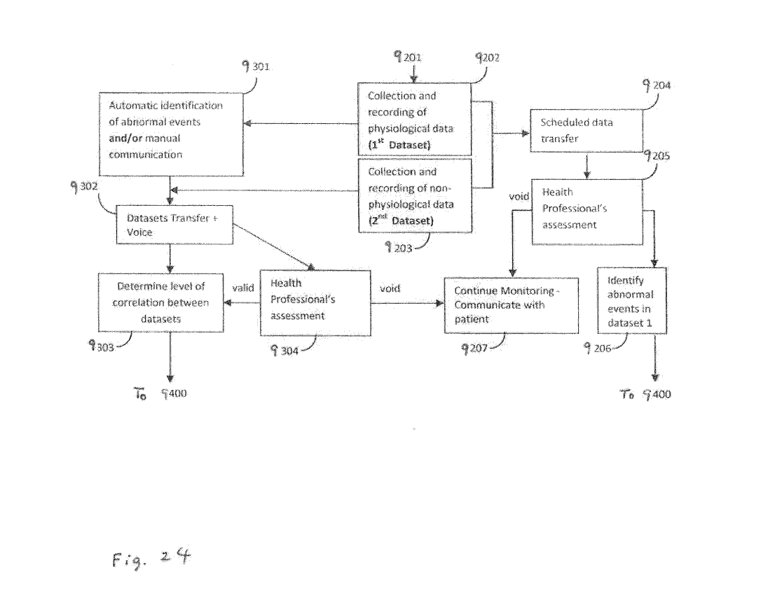

FIG. 23 illustrates a diagram showing a procedure for monitoring, assessing, and processing the physiological and non-physiological information,

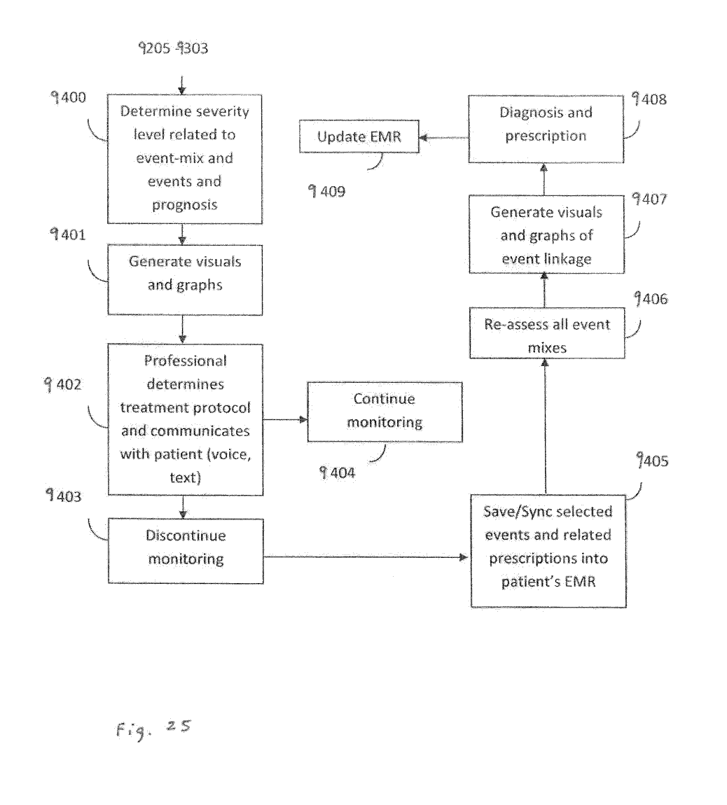

FIG. 24 illustrates a diagram, according to another embodiment, showing a procedure for monitoring, assessing, and processing the physiological and non-physiological information, and

FIG. 25 illustrates a diagram, according to another embodiment, showing a procedure for monitoring, assessing, and processing the physiological and non-physiological information.

FIGS. 1 to 25 have similar parts. The similar parts have the same reference number or same name. The description of the similar parts is included by reference.

FIG. 1 depicts an embodiment of a Universal Body Area Network. The Body Area Network is also called a Body Sensor Network. FIG. 1 shows a Universal Body Area Network 10 that comprises a plurality of Universal Body Unit Sensors (u-BUS) 12, 13 and 14. The Universal Body Unit Sensors 12, 13 and 14 are also called sensor units.

The sensor units 12, 13 and 14 are connected to a sensor hub 3000. The sensor hub 3000 is also called a transceiver. The sensor hub 3000 is connected to a wrist monitor 4000 via a communication link 3400 whilst the wrist monitor 4000 is connected to a cell phone 5000 via a communication link 4500. The sensor hub 3000 and the cell phone 5000 are connected to a control centre 17 via a communication link 5600. The cell phone 5000 is also called a cellular phone or a mobile phone. The control centre 17 is also called a monitoring centre.

The sensor units 12, 13 and 14, the sensor hub 3000 and the wrist monitor 4000 may be placed on the body of a living individual, such as a patient 18. In some cases, the sensor 1A00 or 1B00 may be placed inside the patient 18.

The sensor hub 3000 has a power supply, power management tools and a CPU (central processing unit) unit that is connected to a set of USB ports, to Radio Frequency receiving and emitting channels and to memories that includes RAM (Random Access Memory) as well as Flash memory with relevant database software, relevant files for device management and relevant reference files. The sensor hub 3000 may also contain an embedded database specific circuit for rapidity and efficiency purpose. The specific circuit is not shown in FIG. 1. The CPU unit includes a microprocessor.

Referring to the sensor units 12, 13 and 14, the sensor unit 12 includes a sensor 1A00 which can be physically connected to a sensor module 2A00 via a communication link 1200. The sensor unit 12 is connected to the sensor hub 3000 via a communication link 2300.

Similarly, the sensor unit 13 comprises another sensor 1A00 that can be physically connected to a sensor module 2B00 via another communication link 1200. The sensor unit 13 is connected to the sensor hub 3000 via a communication link 2310.

The sensor unit 14 comprises a sensor 1B00 that is physically connected to a sensor module 2C00 via a communication link 1220. The sensor unit 14 is connected to the sensor hub 3000 via another communication link 2310.

Referring the control centre 17, the sensor hub 3000 is connected to a monitoring station 7000 of the control centre 17 via a communication link 3700. The monitoring station 7000 is also called a control unit. The monitoring station 7000 is connected to a communication station receiver and emitter 6000 via a communication link 7600. In addition, the monitoring station 7000 is also connected to a medical portable device 9000 via a communication link 7900 and to an electronic medical records (EMR) data repository 8000 via a communication link 7800.

In a generic sense, multiple sensor units 12, instead of one sensor unit 12, can be connected to the sensor hub 3000. Similarly, multiple sensor units 13 or multiple sensor units 14 can be connected to the sensor hub 3000.

The cell phone 5000 can be connected to multiple wrist monitors 4000. The cell phone 5000 may be physically attached to the body of the patient 18 via a wrist band.

Moreover, the sensor hub 3000 may be connected to the cell phone 5000. If the wrist-monitor 4000 malfunctions, the sensor hub 3000 can still be in communication with the monitoring station 7000 via the cell phone 5000.

The Universal Body Area Network 10 is intended for monitoring certain parameters of the patient 18.

Referring to the sensor unit 12 or 13, the sensor unit 12 or 13 is intended for collecting information from a living human body of the patient 18. The sensor 1A00 comprises a sensing element that is associated or supported by a number of electronic circuits.

The sensing element includes electrodes, such as ECG (electrocardiogram) or EEG (electroencephalographic) electrodes, or multi-electrodes sensors. The electrodes or multi-electrodes sensors transform a chemical reaction or physical signal into electrical current, voltage or capacitance. The sensing element receives an input electronic signal of a certain electrical current, voltage and frequency and it produces output electronic signal of a certain electrical current, voltage and frequency. The output electronic signal reflects characteristic of the environment that the sensing element is in contact with.

As one example, temperature is determined by measuring a resistance value of the sensor 1A00. An electrical current I is captured by the sensor 1A00 whilst a voltage V of the sensor 1A00 is measured. By using Ohm's law, a resistance value of the sensor 1A00 is determined. The resistance value can then be used to obtain temperature readings using a relationship between the resistance value and the temperature.

In a general sense, the temperature reading is one type of parametric reading. The parametric reading can be related to physiology or non-physiology. The physiological parameters may include cardiac signals, for example ECG signals or heart rhythm, blood pressure, body temperature, body motion status, or biochemical data. The non-physiological parameters may include air temperature, air pressure, or air humidity level.

The electronic circuits allow or enable analog-to-digital treatment or conversion. This conversion is also called analog to digital conversion, which enables an accurate signal processing and translating of sensor reading into useable information.

The sensor module 2A00 or 2B00 provides appropriate voltage and electrical current to activate the sensor 1A00 to collect desired information, as described above.

The collected information may be transferred by the communication link 1200 from the sensor 1A00 to the sensor module 2A00 or 2B00. The communication link 1200 may include two or four wires. As one example, the two wires are connected to a set of ECG (electrocardiogram) electrodes wherein one wire is used for receiving ECG signals and another wire is used for isolating electrical signals. In another example, the four wires are connected to a semi-implanted glucose sensor or to another physiological parameter sensor.

The sensor module 2A00 or 2B00 then treats the collected information. The treatment converts the collected analog information into a digital format. The treatment can also apply fudge values or offset values to calibrate the collected information. The fudge values are intended to eliminate or reduce measurement errors and enhance measurement accuracy. The digital data can also be converted to a biological, electrophysiological, or physical value.

The sensor module 2A00 later sends the received measurements to the sensor hub 3000 via the communication link 2300 using a pre-determined format that is shown in FIG. 2.

The communication link 2300 transfers the measurement via a wired USB (Universal Serial Bus) D+ and D− data connections using USB format. The physical wired USB connection also provides power supply to the sensor module 2A00 that is connected to the USB communication link 2300. The power supply is also be used to power the sensor 1A00.

Similarly, the sensor module 2B00 sends the received measurement to the sensor hub 3000 via the communication link 2310 using a pre-determined data exchange protocol, such as RS 485, ANT+, or other appropriate format. The communication link 2310 may use a wireless USB protocol or other wireless protocol.

Referring to the sensor unit 14, the sensor 1B00 is intended for measurement of environmental or biological information, which requires a more complex initial treatment as compared to information collected from the electrodes or the multi-electrodes sensors of the sensor 1A00.

The sensor 1B00 may detect body motion status, acceleration, geographical position, blood pressure, or other physiological or biological parameters. This can be done inside or outside of a living human body. These functions can be implemented through sensors, which may exist currently in the market, or which still need to be developed. The sensor 1B00 has a specific response curve as well as a pre-determined activation or calibration and measurement protocols. The sensor 1B00 has also a specific response curve as well as a pre-determined activation or calibration and measurement protocols. Moreover, the sensor 1B00 has a sub-module that acts as a direct sensing part that is associated with a companion electronic circuit.

For example, the sensor 1B00 can measure insulin for sensing glucose level of diabetes patient or monitors cardiac activity in real time for pacemakers and defibrillators. The sensor 1B00 can also be used at blood point of use for portable blood laboratories.

The sensor module 2C00 is used for applying a specific flow of electrical conditions necessary for initialization, calibration and measurement of the sensor 1B00.

In addition, the sensor module 2C00 uses an appropriate communication protocol to collect information from the sensor 1B00. The communication protocol includes the SPI (Serial Peripheral Interface) protocol, the I2C (Inter-Integrated Circuit) protocol, or the UART (Universal Asynchronous Receiver/Transmitter) protocol. Then, the sensor module 2C00 treats the collected data or information. The treatment converts the collection information into its digital format for easier communication. The treatment may also be converted the digital value into its biological, electrophysiological, or physical value. The sensor module 2C00 afterward sends the treated information to the sensor hub 3000 via the communication link 2310. The sensor module 2C00 may include means to provide electrical power to the sensor 1B00.

In a broader sense, the sensor module 2A00, 2B00, or 2C00 can serve different types of sensors. While serving different sensor types, the sensor module 2A00, 2B00, or 2C00 produces a standard pre-determined output for the sensor hub 3000. Thus, different sensor type can be connected to the sensor hub. The sensor hub 3000 does not need to be changed when new sensors are introduced for connecting to the sensor hub 3000.

The sensor hub 3000 provides central functions of the Body Area Network 10 and it is intended for receiving information from the sensors modules 2A00, 2B00 and 2C00 by means of associated physical receptor using wired USB, wireless USB communication protocol, or other wireless protocols.

The sensor hub 3000 also sends sequencing instructions to the sensor modules 2A00 via the wired communication link 2300 and to the sensor module 2B00 or 2C00 via the wireless communication link 2310. The sequencing information includes frequency of obtaining sensor information from the sensor 1A00 or 1B00.

The sensor hub 3000 receives initial data and specific associated program for monitoring activity of the sensor 1A00 and 1B00 from the control unit 7000 during an initial setup of a monitoring process via the physical communication link 3700 using the communication protocol USB 2.0 or higher. The control unit 7000 can also update and maintain sensor hub software during the initial setup.

In addition, the sensor hub 3000 treats information collected by the sensors 1A00 and 1B00 by organizing the results or information in a data set, which may be in HL7 (Health Level Seven) standard format. The sensor hub 3000 can also generates periodical summary of the information for displaying by the wrist monitor 4000. As one example, a sensor hub program can reconstruct ECG signals from the wired or wireless ECG related sensor modules.

The sensor hub 3000 may then automatically detect anomalies, errors and missing functions of the treated information.

The sensor hub 3000 have an embedded an RF (radio frequency) module that transmits alarm messages and periodical summary of the collected sensor data of the patient 18 to the wrist monitor 4000. A sequencing program downloaded during the initial sequence defines a frequency of producing data set or datasets.

The alarm, other messages and the datasets are transmitted via the communication link 3400 to the wrist monitor 4000 using Bluetooth wireless communication protocol for providing RF signal exchange.

Referring to the wrist monitor 4000, the wrist monitor 4000 provides means for communicating externally with a qualified professional at the monitoring station 7000. The wrist monitor 4000 also has an alarm module for the patient 18 or his immediate entourage. The wrist monitor 4000 can also display recorded information, summaries in graphic or numeric form, or both in real time.

The wrist monitor 4000 receives monitored data files from the sensor hub 3000 in a predefined format at platform initialization.

The wrist monitor 4000 then relays the data files to the cell phone 5000 via the communication link 4500. The data files include data of the patient 18 and a summary of certain information of the patient 18. The communication link 4500 uses the 3G (Third Generation) or 3.5 G communication protocol for exchanging the data files using RF signals. The data files are intended for later transmission to the monitoring station 7000 by the cell phone 5000.

In addition, the wrist monitor 4000 displays the summary of the measurements, sensor network status, network status alarm and other preset functions.

The cell phone 5000 provides a communication link between the wrist monitor 4000 and the receiving communication station 6000 via the communication link 5600. The communication link 5600 is part of cellular phone network and it uses the 3G (Third Generation) or 3.5 G communication protocol for RF signal exchange. The communication is used to send a data set from the wrist monitor 4000 to the monitoring station 7000 for further data treatment or monitoring.

The communication station receiver and emitter 6000 later send the data set to the monitoring station via the communication link 7600. The communication link 7600 may include a communication protocol, such as IEEE 802.1.1 or a standard LAN (Local Area Network) or wired USB connection.

The monitoring station 7000 is used to initialize the sensor hub 3000 for each patient 18 to whom the device has been indicated or attached for predefined period of time. This initialization is performed prior to the Universal Body Area Network 10 been installed on the patient 18. The initialization is conducted via the communication link 3700.

After the initialization and during patient monitoring, the monitoring station 7000 receives constantly from the wrist monitor 4000 a flow of information that uses the HL7 format.

The monitoring station 7000 includes a computer with sufficient computation power and data storage capabilities to support an installation of a dedicated Body Area Network monitoring program.

The monitoring program treats directly alerts or alarms and data files that are sent by the wrist monitor 4000 in order to issue necessary diagnostics and presentation of data for later analysis by relevant medical authority that may include nurses or doctors.

When the wrist monitor 4000 issues an anomaly or alarm to the monitoring station 7000, the alarm is confirmed or verified by monitoring program and then it can be transmitted to the relevant medical authority by email, pager or cell phone.

The monitoring station 7000 also downloads and uploads relevant information from the medical record data repository 8000 via the communication link 7800 for supporting the monitoring of the patient 18. The communication link 7800 comprises a physical or wireless Local Area Network connection. This information from the medical record data repository 8000 can be used to treat the alerts and the data files from the wrist monitor 4000.

In addition, the monitoring station 7000 is in communication with the medical portable device 9000 via the communication link 7900, which provides a physical USB connection. The medical portable device can be used by qualified personnel to support adequate and to enhance accuracy of clinical decision-making processes for the patient 18.

FIG. 2 shows an example of a format of information 20 that the sensor module 2A00 sends to the sensor hub 3000.

The information 20 includes patient identity information 22, sensor identity information 23, as well as date and time information 24 with corresponding sensor output data values 25. In addition, the information 20 also includes reference date and time 26 and termination information 27. The reference date and time 26 relates to activation or start of the sensor module 2A00 whilst the termination information 27 relates to end of sensing data.

The information 20 is intended for sending in a periodical manner to the transceiver 3000. The frequency of sending is defined during the initialization of the sensor module 2A00.

In a general sense, the sensor module 2B00 or 2C00 of FIG. 1 can send information in a similar format to the sensor module 2B00 or 2C00.

FIG. 3 depicts a further embodiment of the Universal Body Area Network of FIG. 1. FIG. 3 shows a further Universal Body Area Network 30 that has parts that are similar with parts of Universal Body Area Network 10 of FIG. 1.

The Universal Body Area Network 30 has multiple Universal Body Unit Sensors (u-BUS) rather than one u-BUS being connected to the transceiver 3000 of FIG. 1. This depicts an advantage of scalability as number of connected u-BUS can be easily changed and is not restricted to a fixed number.

The Universal Body Area Network 30 has multiple Universal Body Unit Sensors (u-BUS) 12, which are connected to the transceiver 3000 via the wired communication link 2300. One u-BUS 13 and one u-BUS 14 are also connected to the transceiver 3000 via the wireless communication links 2310.

The transceiver 3000 is connected to the wrist monitor 4000 via the communication link 3400. The u-BUS 12, 13 and 14 as well as the transceiver 3000 and the wrist monitor 4000 are provided for the body of the patient 18. The wrist monitor 4000 is connected to the cell phone 5000 by the communication link 4500. The cell phone 5000 is placed in a pocket 32 of the patient 18.

The cell phone 5000 is connected to the monitoring center 17, which can be located in a hospital or a clinic. The monitoring center 17 is used for monitoring the patient 18 and is operated by qualified personnel.

FIG. 4 shows an embodiment of the Universal Body Unit Sensor (u-BUS) 12 of FIG. 1 that has a wired interface. The u-BUS 12 comprises the sensor 1A00 and the sensor module 2A00 that is connected to the sensor 1A00 via the communication link 1200.

The sensor module 2A00 includes a microcontroller 35 that is electrically connected to a sensor analog driver 36, to an analog-to-digital converter 37 and to a communication module 39. The sensor module 2A00 also includes a clock 38. The sensor analog driver 36 is electrically connected to the sensor 1A00 via electrical connections 44 whilst the analog-to-digital converter 37 is electrically connected to the sensor 1A00 via electrical connections 45. The communication module 39 may include a wired USB connection.

The microcontroller 35 comprises a processor, an EEPROM (Electronically Erasable Programmable Read-Only Memory) 40, a SRAM (static random access memory) 41 and a sensor ID (identifier) 42. The sensor ID 42 is stored in a ROM (Read Only Memory) of the microcontroller 35. The processor and the ROM are not shown in the FIG. 4.

During operations, the microcontroller 35 initially sends sensor ID information to the sensor hub 3000 of FIG. 1 via the communication module 39. This information is used by the sensor hub 3000 for identifying the sensor module 2A00 and for selecting appropriate sensor parameters and program for the sensor module 2A00. The sensor parameters and the program are used for managing the sensor 1A00 of the sensor module 2A00. The sensor parameters and the program can originate from the monitoring station 7000.

The microcontroller 35 later receives the selected sensor parameters and the selected program from the sensor hub 3000 via the communication module 39. The sensor parameters and program are then loaded or stored in the EEPROM 40 or the SRAM 41. The EEPROM 40 or the SRAM 41 can have an embedded database to store the sensor parameters for higher data retrieval performance. The microcontroller 35 also stores any initial calibration constant of the sensor parameter in the SRAM 41 or the EEPROM 40. As one example, the initial calibration comprises a reference voltage for ECG electrodes.

The program comprises a firmware for operating or managing the sensor 1A00. The management provides operating conditions of the sensor 1A00 and obtains data from the sensor 1A00 at a certain frequency.

The sensor parameter may include sensor data to biological, electrophysiological, or physical value relationship. Further, the sensor parameters may include sensor characteristics, such as sensor supplier information, sensor manufacturing lot information and sensor type information. The sensor parameters may be obtained from sensor specification that is provided by sensor manufacturer. The sensor information may also be adapted by the monitoring station 7000 for the patient 18.

The monitoring station 7000 can also send patient ID (identifier) information to the microcontroller 35. The patient ID information may be obtained from the electronic medical records (EMR) data repository 8000 of FIG. 1. The sensor parameters, the program and the patient ID information are then stored in non-volatile memory of the EEPROM 35.

The sensor analog driver 36 later provides appropriate electrical current and voltage to drive or power the sensor 1A00 via the electrical connections 44. The electrical current and voltage are controlled by the microcontroller 35 based on the stored sensor parameters.

Afterward, the sensor 1A00 collects information from the patient 18 and transmits the collected patient information in an analog format to the analog-to-digital converter 37 via the electrical connections 45. A device for the amplification that is not shown in FIG. 4 can amplify the collected patient information before the collected patient information reaches the analog-to-digital converter 37. Certain signal requires amplification to reduce distortion by noise that is present in the analog-to-digital converter 37.

The analog-to-digital converter 37 then converts the analog patient information into its digital format and sends it to the microcontroller 35.

The microcontroller 35 then converts the digital data to a biological, electrophysiological, or physical value using the sensor parameter. The conversion can use a simple offset or a more complex response curve.

The microcontroller 35 also organises the converted digital patient information in a pre-determined format as directed by the program and may include data and time information from the clock 38 in the patient information. This organized information is then stored in the SRAM 41 or EEPROM 40 for later transmission by the communication module 39 to the wrist monitor 4000 of FIG. 1.

For securing the stored information during transmission, the transmitted information includes the sensor ID information to distinguish the transmitted information from information of other patient of other sensor network. The transmitted information can also include the patient ID information in addition to the sensor ID information for extended security.

In a generic sense, the sensor module 2A00 can be adapted to support different types of sensor. The adaption is accomplished by loading a specific firmware and specific sensor parameters of the different sensor into the EEPROM 40 or the SRAM 41. The specific firmware and the specific sensor parameter are appropriate for a sensor that is connected to the sensor module 2A00.

The sensor module 2A00 may receive different range of sensor output values but is able to produce a standardised pre-determined output to the sensor hub 3000. Further, new sensors can be easily integrated into the sensor module 2A00. Usually, only specific firmware of the new sensors need to be loaded or installed into the sensor module 2A00 for the sensor module 2A00 to work with the new sensors.

In a further embodiment, the communication module 39 includes a wireless RF communication module instead of a wired USB connection, as shown in FIG. 5. The wireless RF communication module can use wireless USB, SPI, or Bluetooth protocol.

FIG. 6 shows an embodiment of the transceiver 3000 of the Universal Body Area Network 10 of FIG. 1. The transceiver 3000 is connected to the sensor unit 12 via a wired connection and is connected to the sensor unit 13 via a wireless connection.

The transceiver 3000 includes a set of communication ports, a microprocessor 51, a flash memory 52 and a flash to USB microcontroller 53. In addition, the transceiver 3000 also includes a power management circuit 54 and a power supply battery 55. The flash to USB microcontroller 53 is connected to a USB connection 59 whilst the flash memory 52 includes a firmware for controlling internal electronic devices.

The communication ports include a USB communication port 57, a RF reception port 58, the USB connection 59 and a RF emission node 60. Referring to FIGS. 1 and 6, the USB communication port 57 is connected to the sensor unit 12 whilst the RF reception port 58 is connected to the sensor unit 13. The USB connection 59 is connected to the monitoring station 7000 whilst the RF emission node 60 is connected to the wrist monitor 4000. The connections are provided such that a communication between the respective modules can be established.

During an initial start-up, the USB connection 59 receives an initial dataset and a specific program from the control unit 7000 of FIG. 1 using USB protocol. The initial data and the program are intended for monitoring or controlling the sensor units 12 and 13. The program includes an operating system or firmware with specific software for maintaining the transceiver 3000.

The flash to USB microcontroller 53 then transfers the data and the program to the database of the flash memory 52 for storage. This transfer also allows easy installation or upgrade of new firmware. The flash memory 52 provides temporary storage space for the microprocessor 51.

The microprocessor 51 then executes the program in the flash memory 52. In most cases, the program initially checks for existence of sensor units that are connected to the transceiver 3000. After this, the program proceeds to initialize the detected sensor units. Initialization is intended to apply operating conditions to the sensor units. Then, the sensor units may be calibrated for its specific application. The calibration is specific to sensor type. In a special case, the calibration is specific to the sensor unit 12 or 13. The sensor units 12 and 13 are then ready for acquiring sensor data.

As provided in this example, the program in the flash memory 52 initializes the sensor unit 12 via the USB communication port 57. The USB communication port 57 provides a wired two-ways communication means with the sensor unit 12 for receiving sensor data.

Likewise, the program initializes the sensor unit 13 via the RF reception port 58. The RF reception port 58 provides a wireless two-ways communication means with the sensor unit 13 for receiving sensor data.

Afterwards, the USB communication port 57 provides a wired two-ways communication means with the sensor unit 12. It also receives sensor data from the sensor unit 12. Likewise, the RF reception port 58 provides a wireless two-ways communication means with the sensor unit 13 and it also receives sensor data from the sensor unit 13.

The microprocessor 51 then executes the program to organize or convert the received sensor data to HL7 (Health Level Seven) format. In addition, the microprocessor 51 produces periodical summary of the sensor data for sending to the wrist monitor 4000 of FIG. 1 via the RF emission node 60 using Bluetooth communication protocol. The program also determines frequency of generating the periodical summary.

The microprocessor 51 also checks for anomalies, errors and missing information of the received sensor data. Any detected anomalies, errors or missing information can trigger alarm messages that are later transmitted to the wrist monitor 4000 via the RF emission node 60 and to the monitoring center 7000 via a cellular phone network.

FIG. 7 depicts an embodiment of the wrist monitor 4000 of the Universal Body Area Network 10 of FIG. 1.

The wrist monitor 4000 comprises an antenna 65 that is connected to a SIM (Subscriber Identity Module) card module 66 and to a RF transceiver module 67. The SIM card module 66 and the RF transceiver module 67 are connected to a main controller 69 whilst the main controller 69 is connected to a sensor USB hub 71, to a memory module 72 and to an application processor 73.

The SIM card module 66 is connected to audio codecs (compression or decompression) transceivers 75 that are connected to a speaker 76 and to a microphone 77.

The main controller 69 is connected to the memory module 72 via an encryption module 79. The main controller 69 has sufficient computation capacity to execute a program that is stored in the memory module 72. The sensor USB hub 71 is connected to a temperature sensor input 82, to an air pressure sensor input 83, to an altitude sensor input 84 and to other sensor inputs 85. The application processor 73 is connected to a display controller 87.

Also, the wrist monitor 4000 includes a power supply 89 and a synchronization module 90 that is connected to the main controller 69.

In an operational mode, the RF transceiver module 67 can receive patient sensor information from the transceiver 3000 via the antenna 65. The information is then sent to the main controller 69 to verify or to check for abnormal values or events. After this, the information is encrypted by the encryption module 79 for storing in the memory module 72. The encryption module 79 uses Advanced Encryption Standard (AES) protocol for encrypting the data.

If the information includes a patient alarm, the information can be sent to the application processor 73 for displaying continuously to the patient 18 or to a caregiver of the patient 18 via the display controller 87.

The main processor 69 can also use the application processor 73 to display any alarm or to sound the alarm using the speaker 76 via the SIM card module 66 and the audio codecs transceivers 75. The audio codecs transceivers 75 compress or decompress the audio alarm or signal.

The SIM card module 66 provides communication with the monitoring station 7000 of FIG. 1. The communication includes an audio communication between the patient 18 and a user of the monitoring station 7000 using the speaker 76 and the microphone 77.

In addition, the communication may include data exchange between the monitoring station 7000 and the main controller 69. As one example, this data exchange enables a user of the monitoring station 7000 to receive patient information from the wrist monitor 4000 and to monitor remotely conditions of the patient 18. It also enables the user of the monitoring station 7000 to issue alarms or messages to the wrist monitor 4000 using the display or the speaker 76 of the wrist monitor 4000. In certain cases, it is possible to send short text messages and alarms from the monitoring station 7000 to the wrist monitor 4000.

The USB hub 71 communicates sensor data between the main controller 69 and the sensor inputs 82, 83, 84 and 85. The sensor inputs 82, 83, 84 and 85 receive sensor data from sensor modules 2A00, 2B00 and 2C00 of FIG. 1 that are connected to the sensor hub 3000. The sensor data may include environmental and other non-physiological parameters, such as temperature readings that are collected through the temperature sensor input 82 or environmental air pressure readings that are collected through the air pressure sensor input 83. In addition, the sensor data may include environmental altitude readings that are collected through the altitude sensor input 84.

The power supply 89 produces electrical power for electrical devices or modules of the wrist monitor 4000.

FIG. 8 depicts an embodiment of a communication flow chart 95 of the Universal Body Area Network 10 of FIG. 1.

The communication flow chart 95 includes a step 97 of initializing the Universal Body Area Network 10 by the transceiver 3000. The step 97 also includes the transceiver 3000 sending calibration constants of the sensor 1A00 or 1B00 to the sensor module 2A00, 2B00, or 2C00. The sensor module 2A00, 2B00, or 2C00 then applies operational conditions to the sensor 1A00 or 1B00 in a step 98. The operational conditions can be obtained or derived from the calibration constants. The operational constants include electrical currents or voltages for operating the sensor 1A00 or 1B00.

The sensor 1A00 or 1B00 afterward sends analog signals of voltage, electrical current or capacitance of the patient 18 of FIG. 1 to the sensor module 2A00, 2B00, or 2C00 for conversion to its digital value in a step 100.

After this, the sensor module 2A00, 2B00, or 2C00 sends digital value of voltage, electrical current, or capacitance, sensor reading interval to the transceiver 3000 in a step 101. The digital value can be converted to its biological, electrophysiological, or physical value prior to its trans-mission to the transceiver 3000. The digital value is organized in a structure and format that is shown in FIG. 2.

As one example, an environmental temperature sensor connected to the Body Area Network 10 can send information to the transceiver 3000 regarding average ambient temperature measured during a period of one hour with measurement intervals of one minute. The format of such data exchange follows the sequence of data patient ID 22, sensor ID 23, reference time at activation 26, time or delta time after activation, average temperature 25 and end of measurement time 27.

As a similar example for a body temperature at chest level, a relevant sensor can send information to the sensor hub 3000 with interval of one minute of direct values of temperature that is measured every ten seconds. The format of such exchange can follow, as described earlier, the sequence patient ID 22, sensor ID 23, reference time at activation, time or delta time after activation, temperature measured that is sequence time with temperature measured repeated six times and end of measurement time 27.

In the case of ECG electrode sensor, a similar method can be used. Each electrode sends the ECG potential data set in a predefined interval of five second, as an example. This data set can contain direct value of the measured electrical potential for every millisecond interval.

For practical integration purpose in a relevant garment that is suitable for the patient, the ECG chest electrodes can be combined and treated by the specific sensor electrode module that sends directly the set of electrodes results at the same time.

In a purpose of more compatible standard, the sensor module can contain, in the data file sent to the transceiver 3000, measurement unit, such as Celsius degree for temperature, mV (milli-volt) for ECG, nA (nano-Amperes) for chemical sensor, as well as a sensor fabrication identification and relevant checksum or associated algorithm allowing a check by the receiver software for consistency of information. Such format can be compliant to IEEE 1451 for a transducer with a specific relevant modification.

The same format and communication protocol can be used also with personal weight system or other non-body related complex devices that may be connected to the human Body Area Network.

The transceiver 3000 then reconstructs signal values in datasets, stores them and performs structured information processing, such as events analysis and error detection of the signals in addition to sending alerts or alarms to the wrist monitor 4000 in a step 102.

The wrist monitor 4000 later visualises or displays the selected signals and any alert in a step 103. The visualisation includes displaying information of the signals and showing any alert on a display of the wrist monitor 4000. Furthermore, the wrist monitor 4000 exports or sends information of the signal to the monitoring station 7000.

The transmitted information allows the monitoring station 7000 to monitor conditions of the patient in near real-time, in a step 104. Behind the monitoring station 7000, a qualified professional can confirm pathologic findings and make clinical decisions accordingly. The finding can be detected automatically by the software of the device.

These decisions can include either a voice or short text communication or both with the patient 18 via the wrist monitor 4000 in a step 103. Supporting the decision-making, the physiological and non-physiological data can be compiled through specific algorithms, which should help establish a cause-and-effect relationship between an onset of pathological events and the other data.

The pathological events can include arrhythmias whilst the other data can include patient motion status and ambient temperature. The algorithms can establish a statistical relationship which would help understand the individual variance of these abnormal events and drive appropriate diagnostics and decision-making process.

The wrist monitor 4000 can receive the decisions and the support tools from the monitoring station 7000 to send further information to the transceiver 3000 in the step 103. The further information can be related to calibration constants or to initialization of the Universal Body Area Network 10.

For communication purposes, the Universal Body Area Network 10 or 30 and the transceiver 3000 can use the IEEE 1451 transducer universal format that is adapted or extended for medical devices for exchanging data with each other. Similarly, the transceiver 3000 and the wrist monitor 4000 can use HL7 or XML format for data exchange between each other.

Power supply availability status can be used to determine communication means between the sensor module 2A00, 2B00, or 2C00 and the transceiver 3000.

If power supply is not available at the sensor module 2A00, 2B00, or 2C00, the sensor module can use wired USB connection to receive external power so that it can exchange data with the transceiver 3000.

But, if power supply is available at the sensor module 2A00, 2B00, or 2C00, the sensor module 2A00, 2B00, or 2C00 can use Bluetooth or other wireless protocol or wired USB connection to exchange data with and the transceiver 3000. The power supply may include a button battery.

The transceiver 3000 and the wrist monitor 4000 can use the Bluetooth protocol or other wireless protocol for data exchange with each other. The wrist monitor 4000 and the monitoring station 7000 can use 3G (Third Generation), GPRS (General Packet Radio Service), or GSM (Global System for Mobile communications), depending on availability, for data exchange with each other.

FIG. 9 depicts an embodiment of a schematic diagram 108 of the Universal Body Unit Sensor (u-BUS) 13 of FIG. 1. The u-BUS 13 includes the sensor 1A00 that is connected to the sensor module 2B00.

The sensor 1A00 is connected to an amplifier 110 that is connected to a 16 bits SPI (Serial-Peripheral Interface) ADC (Analog-to-Digital) Converter 114. In addition, the ADC Converter 114 is connected to a microcontroller 115 that is connected to a SRAM (Static Random Access Memory) 117.

The amplifier 110 is connected to an output of the sensor 1A00 and to a reference voltage 112. An output of the amplifier 110 is connected to the ADC Converter 114. The reference voltage 112 receives electrical power from a button battery 113.

An output of the ADC Converter 114 is connected to the microcontroller 115 with SPI and USB interfaces. The microcontroller 115, in turn, is connected to the SRAM 117 that is connected to an RF (radio frequency) emitter 118. The microcontroller 115 has an EEPROM (Electrically Erase-able Programmable Read Only Memory) 116.

The EEPROM 16 stores a firmware and sensor parameters. The firmware is intended to govern or to rule the sensor 1A00. The microcontroller 115 performs instructions of the program to activate the sensor 1A00.

The amplifier 110 later drives or amplifies an output analog signal of the sensor 1A00 and then sends it to the ADC converter 114. The ADC converter 114 later converts the amplified analog signal to its digital format using 16 bits resolution and stores the converted digital value of the signal in the SRAM 117. The microcontroller 115 then converts the digital value to its biological, electrophysiological, or physical value.

After this, the microcontroller 15 includes or tags sensor ID information to the digital value. The RF emitter 118 afterward retrieves the stored values from the SRAM 117 and sends it out in a wireless form.

The reference voltage 112 provides a precise reference voltage for the amplifier 110.

In summary, the embodiment has the advantage that the sensor unit 12, 13 and 14 does not require a specific or special set-up. Moreover, the communication protocols of the embodiments as well as its information structure enable a “plug and Play” type of network. The embodiment allows easy integration of other type of sensor units into the sensor network as well as Point-Of-Care devices, such as blood analysis. The point-of-care refers to near to site of patient care.

FIG. 10 shows an embodiment of the sensor 1A00 of the U-BUS 12 of FIG. 1. The U-BUS 12 includes the sensor 1A00 that is connected to the sensor module 2A00.

The sensor 1A00 comprises a resistance thermometer 125 that is connected to connections leads 127 and 128. The connections leads 127 and 128 are enclosed by an insulator 130 whilst the insulator 130 and the resistance thermometer 125 are enclosed by a sheath 132.

Functionally, the resistance thermometer 125 is intended for measuring temperature of the surrounding environment. The connection leads 127 and 128 act as channels for transmitting electrical currents and voltages to the resistance thermometer 125. The insulator 130 electrically as well as physically isolates the connection leads 127 and 128 from each other whilst the sheath 132 protects the insulator 130 and the resistance thermometer 125 from the surrounding environment.

FIG. 11 depicts a schematic diagram of the sensor 1A00 of FIG. 10. A voltage Vwr is applied between a working electrode WE of the resistance thermometer 125 and a reference electrode RE of the resistance thermometer 125 such that an electrical current Iwr flows through the resistance thermometer 125. By measuring the electrical current Iwr, a resistance of the resistance thermometer 125 can be calculated using the measured electrical current Iwr and the voltage Vwr, in accordance with Ohm's law.

FIG. 12 shows a response graph 135 of the resistance thermometer 125 of the sensor 1A00 of FIG. 10. The response graph 135 shows a relationship between temperatures of the resistance thermometer 125 and its corresponding resistances.

FIG. 13 shows a flow chart 140 of the sensor 1A00 of FIG. 10. The flow chart 140 starts with an initialization phase 142 at initial time t1, wherein the electrodes WE and RE have ground or zero volts.

Then, after about two seconds, the sensor 1A00 enters a calibration phase 143 for determining electrical leakage current at the electrodes RE and WE. For this determination, one volt is applied at the electrode WE and a zero volt is applied at the electrode RE for a duration of about a half second whilst an electrical current Iwr1 at the electrode WE and an electrical current −Iwr1 at the electrode RE are measured. The measured calibration current values are then sent to the sensor module 2A00.

In a similar manner, two volts is later applied at the electrode WE and a zero volt is applied at the electrode RE for a duration of about a half second whilst an electrical current Iwr2 at the electrode WE and an electrical current −Iwr2 at the electrode RE are measured. Afterwards, the measured calibration values are sent to the sensor module 2A00.

Later, the sensor 1A00 enters an activation phase 144 after a certain number of seconds. The activation phase 144 is intended to prepare the sensor 1A00 for acquiring sensor data. In this phase 144, one and a half volt is applied at the electrode WE and a zero volt is applied at the electrode RE.

Afterward, the sensor 1A00 enters an acquisition phase 145. One and a half volt is applied at the electrode WE and zero volt is applied at the electrode RE whilst the electrical current Iwr at the electrode WE is measured. The measured data value is then transmitted to the sensor module 2A00.

Based on the transmitted calibration values and the transmitted measured data value, the sensor module computed the sensor data value.

Following the data acquisition, the sensor 1A00 can enter a standby phase 146. In this phase 146, the electrodes WE and RE have ground voltage.

FIG. 14 shows a further embodiment of the sensor 1A00 of the U-BUS of FIG. 1. The sensor 1A00 of FIG. 14 is intended for measuring blood glucose level. This sensor 1A00 has a working electrode WE and a reference electrode RE. A voltage Vwe can be applied the electrode WE and a voltage Vre can be applied at the electrode RE whilst an electrical current flows at the electrode WE.

FIG. 15 depicts a response graph 150 of the sensor 1A00 of FIG. 14. The response graph 150 shows a relationship between the electrical current Iwe of the sensor 1A00 and its corresponding glucose level.

FIG. 16 shows a flow chart 155 of the sensor 1A00 of FIG. 14. The flow chart 155 has an initialization phase 157. In this phase 157, a ground voltage is applied to the electrodes RE and WE.

Two seconds after initialization, the sensor 1A00 enters a calibration phase 158 in which a ground voltage is applied to the electrode RE and one volt is applied to the electrode WE for a duration of about twenty seconds whilst the electrical current Iwe1 at the electrode WE is measured. The measured calibration current value is later transmitted to the sensor module that is connected to the sensor 1A00 for processing.

Later, in a similar manner, a zero voltage is applied to the electrode RE and two volts are applied to the electrode WE for a duration of about a half second whilst the electrical current Iwe2 at the electrode WE is measured. The measured calibration current value is afterward transmitted to the sensor module.

About twenty seconds after initialization, the sensor 1A00 enters an activation phase 159, wherein ground voltage is applied to the electrode RE and one and a half volt is applied to the electrode WE.

Later, the sensor enters an acquisition phase 160. In this phase 160, an electrical current Ire is applied at the electrode RE and one volt is applied at the electrode WE whilst the electrode RE is maintained at half volt and the electrical current Iwe at the electrode WE is measured. The acquired measured electrical current value is later sent to the sensor module.

The sensor module later determines the sensor data using the transmitted calibration values and the acquired measured value.

Afterward, the sensor 1A00 can enter a standby phase 161 in which the electrodes RE and WE are kept at ground voltage.

FIG. 17 depicts a further embodiment of the Universal Body Area Network of FIG. 1. FIG. 17 shows a Universal Body Area Network 170 that includes the sensor hub 3000 connected to the wrist monitor 4000 of FIG. 1. The wrist monitor 4000 is integrated with the cell phone 5000. As shown here, the wrist monitor 4000 includes the cell phone 5000.

In another embodiment of the Universal Body Area Network of FIG. 1, FIG. 18 shows a Universal Body Area Network 175 that includes the sensor hub 3000 connected to the wrist monitor 4000 of FIG. 1. The wrist monitor 4000 is part of the cell phone 5000. Herein, the wrist monitor 4000 is implemented as an application of the cell phone 5000. The application is a software program, which is loaded into the cell phone 5000.

The above subject matter can be put into practice in the following manner, wherein the system collects and stores cardiovascular and ambient data through a monitoring device, which can be worn by the person to be monitored for a specific medical condition. Data are collected in two sets (physiological and non-physiological). The physiological events are presented to the medical professionals in a visual form using graphics and numbers for assessment. The physiological information can include but not limited to the detection of cardiac arrhythmia events, blood pressure, and respiratory rate.

The operation of the system can include at least one implantable or non-implantable sensor, which collects physiological information. This sensor or electrode can be included in a wearable body area network, which can be wired to a transceiver. The transceiver cumulates the information and transmits it to a wrist wearable device where it is stored. The wrist device can connect to a monitoring centre via cellular network, using an embedded SIM card module. This connection can be bidirectional and can include data and voice.

The wrist unit can store physiological and non-physiological data for as long as 4 weeks of monitoring time. After the end of the monitoring session, these data can be downloaded from the wrist device to the monitoring workstation using standard USB cable.

At least one of the following advantages can be achieved: The system can monitor and record cardiovascular events 24 hours a day for up to 30 days and can transfer data to a monitoring centre at any time for assessment. It enables the medical professional to have a direct and real time access to the data and the context in which the events occurred. Furthermore, the automatic detection of events combined with human validation increase the sensitivity of the method to confirm a diagnosis. This is further enhanced by an event recording over a long period of time, which reduces the false negative probes.

The system can establish a two-way voice and data communication between the monitored person and the healthcare professional at the monitoring instance. The monitoring workstation can be linked to an existing hospital information system and exchange patient data, which can be integrated with the electronic medical records of the patient (EMR).

The wireless body area network functionality can be extended to include an unlimited number of sensors. The information collected by these additional sensors can be categorized, codified (Unique ID), integrated within the data-stream and then transferred to the wrist unit via the transceiver. The software component of the wrist device can process the information coming from all the sensors for further analysis.

The system does identify and determine the impact of that specific event on other physiological parameters or its potential relationship with other non-physiological data (like ambient temperature, altitude, etc) that might have triggered it. Moreover, the systems can avoid a physical connection to a prior art cellular phone or PDA in order to establish communication with the monitoring instance. Accountability-related (medico-legal) issues in case of malfunction are avoided.

FIG. 19 illustrates a system 9103 for recording physiological and non-physiological information from a living individual 9100 and his/her immediate environment. The system 9103 can communicate the information via devices 9101 and 9102 to a monitoring centre 9105.

The system 9103 can include one wearable component of device 9102 connecting at least one external or implantable sensor to a transceiver. The device 9102 and the transceiver can be a Wireless Body Area Network (WBAN). The sensors can collect physiological information from the individual 9100 and transfer it to a transceiver of device 9102. The physiological information captured can be ECG, heart rate, blood pressure, body temperature and respiratory rate and 3D motion status of the individual 9100. This physiological information is referred to in FIG. 23 as first (1st) dataset 9202.

The device 9101 can collect non-physiological information such as ambient air temperature, ambient air pressure, altitude, ambient air humidity level. This non-physiological information is referred to in FIG. 24 as a second (2nd) dataset 9203.

The first dataset 9202 can be then transferred from the device 9102 to the wearable wrist device 9101. The first and second datasets 9202 and 9203 can be recorded on the device 9101 and can be transferred to a monitoring centre 9105.

Prior to monitoring start, the device 9101 can be preloaded with the data of the individual 9100, which can be provided through his/her Electronic Medical Records (EMR) stored at the monitoring centre 9105. This data can include the unique patient ID (identifier), the patient's detailed and current drug prescription list, the ID and contact of the patient's attending/monitoring physician and the address and contact of the monitoring centre. The data can use XML or HL7 formats. The data upload to device 9101 can occur via standard USB cable connecting to the clinical information systems 9106 and 9107. The devices 9101 and 9102 are the two wearable components of one single device 9103.

The wearable wrist device 9101 can directly connect to the monitoring centre 9105 via existing cellular network by using 2.5G or higher communication protocols. The communication can include bidirectional voice and data transmissions as well as remote commands from the monitoring centre to reset the device. Data transmission can include the first and second datasets 9202 and 9203 in one way by the devices 9101 and 9102 via the monitoring centre 9105 and medical prescriptions in the other way by the monitor centre 9105 to the device 9101.

The individual 9100 or patient can view all data at any point in time via the wearable wrist device display.

Data between the wearable wrist device 9101 and the monitoring centre 9105 are encrypted and can be exchanged using standard XML or HL7 protocols.

The operation of the monitoring centre can include a medical professional 9108 operating a dedicated monitoring system with display and data processing of the information system 9106. The monitoring system can exchange specific patient information with the existing clinical Information System 9107. The medical professional 9108 can operate simultaneously on the clinical information system for example to update new drug prescriptions and transfer selected information to the monitoring system, which can in return forward the same to the wearable wrist device 9101. Both systems 9106 and 9107 can exchange information using standard XML or HL7 protocols.

At the end of the monitoring period, data recorded on the wearable wrist device 9101 can be uploaded into the monitoring system for consolidation and processing. Data upload can be done using an USB cable connection between both devices 9106 and 9107.

FIG. 23 shows a step 9200 of a procedure for processing information, which can include unique ID of the patient 9100 of the FIG. 19 and some other case-related data like medicines currently in use by the patient and contact information of the monitoring centre and at least one healthcare professional in charge during the monitoring period. The information can be provided through the clinical information system 9107 of FIG. 19 of the monitoring instance. Data are uploaded to the wearable wrist device 9101 via a standard USB cable connection. The data uploaded into the wrist device 9101 of FIG. 19 erases and replaces pre-existing data from a previous patient. After data upload, a health professional validates the new entry and the new settings for a new monitoring session 9201.

The applications also relates to a system and method for monitoring, assessing and communicating physiological and non-physiological information to increase the accuracy of diagnosis and decision-making for the treatment of cardiovascular conditions. This application is described with reference to FIG. 19 and to FIGS. 23, 24, and 25.

The embodiment describes a system and method for collection, analysis, and two-way communication of physiological and non-physiological information obtained from a living human being and his immediate environment, in real-time. The physiological information can be of two types—subjective and objective. The non-physiological information can be—but is not limited to—ambient temperature, humidity level, altitude, magnetic fields intensity, and atmospheric pressure. All this information is presented to and is partially assessed by humans. An algorithm establishes the statistical significance of linkage between the data and presents visual results to the medical professionals to help decision-making for diagnosis and treatment. The areas of application can be the identification, pathophysiological understanding, and treatment of cardiac arrhythmias in relationship with the person's context of onset. The data are processed in standard XML or HL7 format and can be integrated with the person's electronic medical records.

This application describes a system and method to handle physiological event information and non-physiological data relating to cardiovascular conditions. For example, identifying specific arrhythmia types and establishing a statistical cause and effect relationship with a potential drop of the blood pressure, loss of consciousness and other objectively measured ambient parameters in which these events might occur, like outside temperature, subject's motion status at the time of onset of these events.

These events can be selected and integrated with the patient's electronic medical records using XML or HL7 protocols.

This system collects and stores cardiovascular and ambient data through a monitoring device that can be worn by the person to be monitored for a specific medical condition. Data are collected in two sets, physiological and non-physiological. The physiological events are presented to the medical professionals in a visual form using graphics and numbers for assessment. The physiological information can include but not limited to the detection of cardiac arrhythmia events, blood pressure, and respiratory rate.

The operation of the system can include the data input of subjective information by the monitored person, such as chest pain or palpitations and their duration at any time. This subjective information can be part of the physiological data.

Furthermore, the system can include a bi-way voice communication between the monitored and the monitoring parties via cellular network.

Once the events confirmed by the medical professional, the full data is displayed in a graphic representation including the events themselves and non-physiological data collected around the same time, with their respective duration.

Over an extended monitoring and events' recording session that can last up to 30 days, the system can establish and present a graph on the level of statistical significance between the physiological and non-physiological data.

At least one of the following advantages can be achieved: The system can monitor and record cardiovascular events 24 hours a day for up to 30 days and can transfer data to a monitoring centre at any time for assessment. It enables the medical professional to have a direct and real time access to the data and the context in which the events occurred. Furthermore, the automatic detection of events combined with human validation increase the sensitivity of the method to confirm a diagnosis. This is further enhanced by an event recording over a long period of time, which reduces the false negative probes.

Moreover, analyzing and compiling the physiological and non-physiological datasets by the system a help assess the predictivity and risk level for the potential onset of other events and related complications. This can support medical professionals in the approach of diagnosis and related decision-making coming close to more individualized treatment prescription.

The system enables the medical professional to communicate with the patient via text to send recommendations and/or specific drug and non-drug prescriptions, as well as by voice.

The data exchanged can be stored in XML and/or HL7 format. They may also include a unique patient identifier that can be loaded from the clinical information system before the monitoring commences. All data can be specifically selected and be integrated with the patient's electronic medical records.

The application can also be described with the following list of elements and features:
a device-related method embedding: collecting and processing physiological (ECG, heart rate, blood pressure, motion status) and non-physiological information from a living individual and his environment identifying and monitoring pathological events, such as cardiac arrhythmias and/or high blood pressure, a bi-way Communication that includes data exchange and voice with the monitoring centre,
a visual representation of events' characteristics including incidence and duration,
a visual representation of events' characteristics related to the non-physiological information on a time scale,
a method to determine and to present graphically the statistical significance of cause-and-effect relationship between the physiological events on the one hand and the physiological and non-physiological events on the other hand,
a method to determine and graphically represent the potential trend of physiological events over a time scale, the relative morbidity risk level all related to the non-physiological events surrounding the individual, and
a method to communicate and exchange data with existing other systems using standard XML and/or HL7 data formats.

One embodiment provides a method for analyzing the signals for achieving a diagnosis and sending a message and an apparatus for using the method. One embodiment focuses on an apparatus for signal pick-up amplification and processing. The measurements are analyzed on the flight and are handed over to a higher messaging instance. A sensor module is provided for that. It does in principle not matter whether one measures heart data, insulin data or any other data, the module is forwarding this data within a wireless body-network. The net is standardized so that several modules can be integrated.

The embodiments also measure environmental data such as humidity, temperature, height above or below sea-level, so that any pathological event which may occur can be matched with the environmental data. Arrhythmias can therefore be matched with a certain height, position, or location (GPS) of the patient. Walking speeds of the patient can be traced or external electric/magnetic fields.

All data that are transmitted from the patient to the hospital or health care center/organization can be immediately imported into an electronic file (patient's electronic medical record, EMR). In the same manner, data can be exported to the patient from the electronic file, which is kept at the hospital or health care center/organization. This enables the hospital or health care center/organization to transmit a medical recommendation to the patient, especially via SMS messaging or via email. The recommendation may include an advice on a drug dosage.

The following itemized description serves to provide an overview.
1. Apparatus for collecting and processing physiological (ECG, heart rate, blood pressure, motion status) and non-physiological information from a living individual and its environment and for recording, identifying and monitoring pathological events such as cardiac arrhythmias or high blood pressure.
2. Apparatus according to item 1, being provided with storage means for continuous recording for at least 4 weeks time.
3. An integrated GSM/GPRS module to establish bi-way data and voice communication with the monitoring centre and health professionals, using standard cellular phone network and preferably dedicated priority servers.
4. The GSM/GPRS module of item 3 wherein being provided with a link means with the monitoring centre, the link means provided for avoiding interferences with other calls/communicating devices.
5. Method for data encryption and transmission using Advanced Encryption Standards (AES).
6. A system for determining and graphically representing the potential trend of physiological events over a time scale, the relative morbidity risk level being related to the non-physiological events surrounding the individual.
7. A system for storing, exchanging, and viewing data with and from other systems using standard XML and/or HL7 data formats. Data include but nor limited to Patient Demographics, physiological/non-physiological events and medication information.
8. A system for retrieving data from and monitor multiple patients simultaneously from the monitoring centre.

### REFERENCE NUMBERS

10 Body Area Network
12 sensor unit
13 sensor unit
14 sensor unit
17 control centre
18 patient
20 information
22 patient ID information
23 sensor ID information
24 date and time information
25 sensor output data values
26 reference date and time
30 Universal Body Area Network
32 patient pocket
35 microcontroller
36 sensor analog driver
37 analog-to-digital converter
38 clock
39 communication module
44 electrical connections
45 electrical connections
40 EEPROM
41 SRAM
42 sensor ID
44 electrical connections
51 microprocessor
52 flash memory
53 flash to USB microcontroller
54 power management circuit
55 power supply battery
57 USB communication port
58 RF reception port
59 USB connection
60 RF emission node
65 antenna
66 SIM card module
67 RF transceiver module
69 main controller
71 sensor USB hub
72 memory module
73 application processor
75 audio codecs transceivers
76 speaker
77 microphone
79 encryption module
82 temperature sensor input
83 air pressure sensor input
84 altitude sensor input
85 sensor inputs
87 display controller
89 power supply
90 synchronization module
95 flow chart
97 step
98 step
100 step
101 step
102 step
103 step
104 step
108 schematic diagram
110 amplifier
112 reference voltage
113 button battery
114 ADC Converter
115 microcontroller
116 EEPROM
117 SRAM
118 RF emitter
125 resistance thermometer
127 connections lead
128 connections lead
130 insulator
132 sheath
135 response graph
140 flow chart
142 phase
143 phase
144 phase
145 phase
146 phase
150 graph
155 flow chart
157 phase
158 phase
159 phase
160 phase
161 phase
170 Body Area Network
175 Body Area Network
1200 communication link
1220 communication link
2300 communication link
2310 communication link
3000 sensor hub
3400 communication link
3700 communication link
4000 wrist monitor
4500 communication link
5000 cell phone
5600 communication link
6000 communication station receiver and emitter
7000 monitoring station
7600 communication link
7800 communication link
7900 communication link
8000 medical records data depository
9000 medical portable device
9103 system
9100 individual
9101 device
9102 device
9105 monitoring centre
9106 information system
9107 information system
9108 medical professional
9202 dataset
9203 dataset
1A00 sensor
1B00 sensor
2A00 sensor module
2B00 sensor module
2C00 sensor module

1. A sensor module for a patient on-body sensor, the sensor module comprising
a device for providing electrical power to a patient on-body sensor,
a converter means for converting an analog patient on-body sensor signal to a digital signal,
a storage means for storing a patient on-body sensor identifier information and for storing the digital signal,
a determining means for determining a parameter of the patient using a value of the digital signal, and
a transmitting means for sending data regarding the parameter and the patient on-body sensor identifier information.

2. A sensor module of claim 1, wherein the transmitting means is provided for sending a patient identifier information.

3. (canceled)

4. (canceled)

5. (canceled)

6. A sensor module of claim 1, wherein the sensor module further comprises a clock for recording a timing of the analog signal.

7. (canceled)

8. A sensor module of claim 1, wherein the transmitting means comprises a means for wired transmission of the data regarding the parameter.

9. A sensor module of claim 1, wherein the transmitting means comprises a means for wireless transmission of the data regarding the parameter.

10. A monitoring device of a Body Area Network comprising
a sensor module of claim 1 and
a sensor that is connected to the sensor module,
wherein the sensor receives electrical power from the sensor module and wherein a storage means of the sensor module comprises a distinguishing sensor identifier information.

11. A sensor hub for receiving a pre-determined signal from at least one monitoring device of claim 10, processing the pre-determined signal, and transmitting the processed signal to a wrist monitor.

12. A sensor hub of claim 11 wherein the pre-determined signal comprises data regarding a parameter of a patient.

13. A Body Area Network that comprises
at least one monitoring device claim 10, and
a sensor hub of claim 11 that is connected to the at least one monitoring device.
wherein a sensor module of the monitoring device sends a distinguishing sensor identifier information for a sensor of the monitoring device to the sensor hub.

14. A Body Area Network of claim 13, wherein the sensor hub comprises a summary means for providing a periodic summary of data regarding a parameter of a patient.

15. A Body Area Network of claim 14, wherein the sensor hub comprises a sending means for sending the periodic summary.

16. A method of operating a sensor module for an on-body sensor, the method comprising
providing electrical power to the sensor,
the sensor transmitting an analog signal of a patient to the sensor module,
converting the analog signal to a digital signal,
storing the digital signal,
determining a parameter of the patient using a value of the digital signal,
incorporating data regarding a sensor identifier into data regarding the parameter of the patient, and
sending the data regarding the parameter of the patient.

17. (canceled)

18. (canceled)

19. (canceled)

20. A method of claim 19, wherein the method further comprises incorporating data regarding a timing of the digital signal into the data regarding the parameter of the patient.

21. (canceled)

22. (canceled)

23. A Body Area Network comprising
with a plurality of monitoring devices of claim 10,
a sensor hub of claim 11,
a monitoring device,
a cell phone, and
a control centre,
the body area network further comprising
a communication link between the sensor hub and the monitoring device, for sending data regarding a parameter of a patient and a patient on-body sensor identifier information from a monitoring device to the sensor hub,
a communication link between the monitoring device and the cell phone, for sending the data from the monitoring device to the cell phone, and
a communication link between the cell phone and the control centre, for sending the data from the cell phone to the control centre.

24. A method for operating a Body Area Network comprising
sending data regarding a parameter of a patient and a patient on-body sensor identifier information from a monitoring device to a sensor hub,
sending the data from the monitoring device to a cell phone, and
sending the data from the cell phone to a control centre.
